(19) United States
(12) Patent Application Publication
(10) Pub. No.: US 2025/0095787 A1
(43) Pub. Date: Mar. 20, 2025

(54) SYSTEMS AND METHODS FOR IDENTIFICATION OF STRUCTURAL VARIANTS BASED ON AN AUTOENCODER

(71) Applicant: MyOme, Inc., Menlo Park, CA (US)

(72) Inventor: Pauline NG, Menlo Park, CA (US)

(21) Appl. No.: 18/959,135

(22) Filed: Nov. 25, 2024

Related U.S. Application Data
(63) Continuation of application No. PCT/US23/23529, filed on May 25, 2023.
(60) Provisional application No. 63/346,096, filed on May 26, 2022.

(51) Int. Cl. G16B 40/30; G06N 3/0455; G16B 20/20; G16B 40/20

(52) U.S. Cl. CPC G16B 40/30; G06N 3/0455; G16B 20/20; G16B 40/20

(57) ABSTRACT

Disclosed herein are systems and methods for evaluating candidate structural variants in the genome of a subject to determine if the structural variant is real. An autoencoder trained on suitable reference samples may be used to encode and then reconstruct a read depth profile for sequencing data from the subject encompassing a candidate structural variant region and a reconstruction error may be determined and used to identify whether the candidate structural variant is real or not. The reconstruction error may be statistically evaluated relative to other test samples analyzed by the trained autoencoder to assess how significantly the subject's reconstruction error differs from the reconstruction errors of reference samples.

### CROSS-REFERENCE TO RELATED APPLICATIONS

This application is a continuation of International Application No. PCT/US23/23529, filed May 25, 2023, which claims the benefit of U.S. Provisional Application No. 63/346,096, filed on May 26, 2022, both of which are incorporated herein by reference in their entirety.

### TECHNICAL FIELD

The disclosure generally relates to genomics. More particularly, embodiments of the disclosure relate to methods and/or systems for recognizing structural variants using a neural network autoencoder.

### BACKGROUND

Structural variants (SVs) are genomic alterations. There may be many different types of structural variation such as deletions, duplications, copy number variants, insertions, inversions and translocations. Structural variants can cause disorders such as genomic syndromes, autism, intellectual disability, ichthyosis, and various other diseases. Hence, identifying SVs is of critical importance. Identification of structural variants within a subject's genome are typically performed manually by visual inspection of genomic data. Such visual inspection is time-consuming, prone to error, and not scalable. Accordingly, there is a need for improved systems and methods for effectively identifying structural variants in a subject's genome.

### BRIEF DESCRIPTION OF THE DRAWINGS

Embodiments of the disclosure are illustrated by way of example and not limitation in the figures of the accompanying drawings in which like references indicate similar elements.

FIG. 1 is a block diagram that illustrates an example of a system architecture, in accordance with one or more embodiments of the present disclosure.

FIG. 2 is a diagram illustrating an example autoencoder that may be used in accordance with one or more embodiments of the present disclosure.

FIG. 3 is a flow diagram illustrating an example process for identifying structural variants, in accordance with one or more embodiments of the present disclosure.

FIG. 4 is a flow diagram illustrating an example process for identifying structural variants, in accordance with one or more embodiments of the present disclosure.

FIG. 5 is a flow diagram illustrating an example process for identifying structural variants, in accordance with one or more embodiments of the present disclosure.

FIG. 6 is a block diagram illustrating an example computing device that may be used in accordance with one or more embodiments of the present disclosure.

FIG. 7A illustrates an example input to an autoencoder, in accordance with one or more embodiments of the present disclosure.

FIG. 7B illustrates an example output generated by an autoencoder, in accordance with one or more embodiments of the present disclosure.

FIG. 8A illustrates an example input to an autoencoder, in accordance with one or more embodiments of the present disclosure.

FIG. 8B illustrates an example output generated by an autoencoder, in accordance with one or more embodiments of the present disclosure.

FIG. 9A illustrates an example input to an autoencoder, in accordance with one or more embodiments of the present disclosure.

FIG. 9B illustrates an example output generated by an autoencoder, in accordance with one or more embodiments of the present disclosure.

### DETAILED DESCRIPTION

Disclosed herein are methods and systems that use machine learning for evaluating potential structural variation in the genome of a subject. As used herein, a “subject” is an organism having a genome within which structural variation may exist. According to some embodiments, the subject is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird. Non-human subjects may be livestock. According to some specific embodiments, the subject is a human. According to some specific embodiments, the subject may have cancer (e.g., the subject may have a tumor which can be sampled for genetic analysis according to the methods described herein). The subject may be a female (e.g., a female human). The subject may be a male (e.g., a male human). In some embodiments, the subject is an adult subject. In other embodiments, the subject is a pediatric subject, such as a neonate, an infant, or a child. In some embodiments, the subject may be an embryo or a fetus. As used herein, an “embryo” may refer to a cellular organism produced by sexual reproduction, including a zygote, morula, and blastocyte, up to the stage of development where the embryo becomes a fetus. An embryo may exist in vitro (e.g., for purposes of IVF) or in utero. As used herein, a “fetus” may refer to an unborn offspring produced by sexual reproduction and existing in utero, beginning at the stage of development where the unborn offspring is no longer characterized as an embryo. Thus, a subject may be considered either an embryo or a fetus from the single cellular stage until the fetus is born. In humans, the offspring is usually considered to be a fetus at approximately 8 weeks following conception. It is well understood in the art what types of genetic material can be effectively obtained from an embryo or a fetus as well as the techniques for doing so and any inherent risks associated therewith.

Structural variation in a subject may be analyzed according to the methods and/or systems described herein based on genetic sequencing data obtained from one or more samples from the subject and genetic data obtained for one or more reference samples (e.g., from a reference population). The samples may be body fluid samples (e.g., a blood sample, a saliva sample) or tissue biopsy samples. The types of samples used to obtain sequencing data may be the same for the subject and the one or more reference samples or may be different. In some embodiments, the reference samples may be partitioned into training samples, test samples, and/or validation samples.

Genetic sequencing data may be obtained from cellular DNA. The cellular DNA may be from cells found within a body fluid (e.g., blood or saliva). In some embodiments, the sequencing of cellular DNA may be performed on blood cells (e.g., white blood cells) or other cells collected through noninvasive or minimally invasive techniques (e.g., cells found in saliva). Sequencing cellular DNA may involve isolating one or more cells from a fetus or embryo according to methods which are well understood in the art. Such approaches typically require invasive techniques that may impose a risk to the embryo or fetus. In some embodiments, cellular DNA used for sequencings may be obtained using non-invasive or minimally invasive techniques, such as blood draw.

In some embodiments, sequencing data may be obtained from cell-free DNA. Cell-free DNA is DNA that is found outside a cell, e.g., freely circulating in the bloodstream or in the cell culture medium of cultured cells, such as embryos grown for in vitro fertilization (IVF). Cell-free DNA may comprise cell-free fetal DNA (cffDNA). Cell-free DNA may comprise circulating tumor DNA (ctDNA). Cell-free DNA may provide a relatively abundant source of genetic material that can be obtained from a non-invasive or minimally invasive procedure, such as sampling cell culture medium or drawing blood from a subject. Cell-free DNA may provide ample genetic information for whole genome sequencing of the subject from whom the cell-free DNA is derived. For instance, shotgun sequencing of cell-free DNA may be used to sequence one or more chromosomes of the subject.

Cell-free fetal DNA (cffDNA) is fetal DNA that circulates freely in the maternal blood. Thus, cffDNA may be obtained from maternal blood sampled, for example, by venipuncture. Analysis of cffDNA is a method of non-invasive prenatal diagnosis that may be ordered for pregnant women. cffDNA originates from placental trophoblasts. Fetal DNA is fragmented when placental microparticles are shed into the maternal blood circulation. Because cffDNA fragments, which are approximately 200 bp in length, are significantly smaller than maternal DNA fragments, they can be distinguished from maternal DNA fragments. Approximately 11-13.4% of the cell-free DNA in maternal blood is cffDNA, although the amount varies widely between pregnant women. cffDNA generally becomes detectable after five to seven weeks gestation and its amount increases as the pregnancy progresses. The quantity of cffDNA in maternal blood diminishes rapidly after childbirth, generally being no longer detectable about 2 hours after delivery. Analysis of cffDNA may provide earlier diagnosis of fetal conditions than other techniques. cffDNA may be analyzed, for example, by massively parallel shotgun sequencing (MPSS), targeted massive parallel sequencing (t-MPS), and SNP assays.

ctDNA is tumor-derived fragmented DNA in the bloodstream that is not associated with cells. Because ctDNA may reflect the entire tumor genome, it has gained traction for its potential clinical utility. “Liquid biopsies” in the form of blood draws may be taken at various time points to monitor tumor progression throughout a treatment regimen. ctDNA originates directly from the tumor or from circulating tumor cells (CTCs), which are viable, intact tumor cells that shed from primary tumors and enter the bloodstream or lymphatic system. The precise mechanism of ctDNA release remains unclear. The biological processes postulated to be involved in ctDNA release include apoptosis and necrosis from dying cells, or active release from viable tumor cells. Studies in both human (healthy and cancer patients) and xenografted mice show that the size of fragmented cfDNA is predominantly 166 bp long, which corresponds to the length of DNA wrapped around a nucleosome plus a linker. Fragmentation of this length might be indicative of apoptotic DNA fragmentation, suggesting that apoptosis may be the primary method of ctDNA release. The fragmentation of cfDNA is altered in the plasma of cancer patients. In healthy tissue, infiltrating phagocytes are responsible for clearance of apoptotic or necrotic cellular debris, which includes cfDNA. cfDNA in healthy patients is only present at low levels, but higher levels of ctDNA in cancer patients can be detected with increasing tumor sizes. This possibly occurs due to inefficient immune cell infiltration to tumor sites, which reduces effective clearance of ctDNA from the bloodstream. Comparison of mutations in ctDNA and DNA extracted from primary tumors of the same patients has revealed the presence of identical cancer-relevant genetic changes, allowing for the possibility of analyzing ctDNA in order to analyze the genetic makeup of tumor cells. Accordingly, ctDNA may be used for earlier cancer detection and treatment follow up monitoring.

Various methods of DNA sequencing are well known in the art and may be used to obtain the sequencing data used by the methods and/or systems described herein unless dictated otherwise, explicitly or by context. DNA sequencing may comprise for example Sanger sequencing (chain-termination sequencing). DNA sequencing may comprise use of next-generation sequencing (NGS) or second-generation sequencing technology, which is typically characterized by being highly scalable, allowing an entire genome to be sequenced at once. NGS technology generally allows multiple fragments to be sequenced at once allowing for “massively parallel” sequencing in an automated process. DNA sequencing may comprise third generation sequencing technology (e.g., nanopore sequencing or SMRT sequencing), which generally allows for obtaining longer reads than obtainable via second generation sequencing technology. Sequencing may comprise paired-end sequencing, where feasible, in which both ends of a DNA fragment are sequenced, which may improve the ability to align the reads to a longer sequencing. DNA sequencing may comprise sequencing by synthesis/ligation (e.g., ILLUMINA® sequencing), single-molecule real time (SMRT) sequencing (e.g., PACBIO® sequencing), nanopore sequencing (e.g., OXFORD NANOPORE® sequencing), ion semiconductor sequencing (Ion Torrent sequencing), combinatorial probe anchor synthesis sequencing, pyrosequencing, etc.

Shotgun sequencing refers to a method of sequencing random DNA strands from a genome or large genetic sample. DNA is broken up randomly into numerous small segments, which are sequenced (e.g., using the chain termination method) to obtain reads. Multiple overlapping reads for the target DNA are obtained by performing several rounds of this fragmentation and sequencing. Computational algorithms then use the overlapping ends of different reads to assemble the reads of the random segments into a continuous sequence. Shotgun sequencing may be used for whole genome sequencing. Any suitable form of sequencing, including those describe herein, may be used to identify variants (e.g., SNPs) in a subject which may subsequently be used as the basis for measuring read depth. In some embodiments, hierarchical sequencing may be used for whole genome sequencing.

### Workflows and Systems for Evaluating Candidate Structural Variants

A workflow for identifying structural variants may use DNA sequencing data from a subject as an input and output structural variants within the analyzed portion(s) of the subject's genome. Structural variant workflows may include aligning sequence reads to a reference genome and then applying one or more structural variant algorithms to identify candidate structural variants which may be evaluated according to the methods and/or systems described herein.

According to conventional structural variant workflows, after a candidate structural variant is identified, the read depth profile of the genomic region of the candidate structural variant is plotted alone, or with other reference samples, and undergoes visual inspection. The visual inspection is used to determine whether the structural variant is polymorphic in the population, a sequencing artifact, or a rare structural variant that could be of clinical interest. A structural variant that is real (e.g., not due to a sequencing artifact or noisy sequencing) and/or rare (e.g., not polymorphic in a reference normal population) is of clinical interest. This visual inspection is time-consuming, prone to error, and not scalable. In addition, the visual inspection is often performed manually (e.g., by a user, clinician, physician, scientist, etc.).

In an effort to reduce the manual burden of visually inspecting these read depth profiles, artificial intelligence methods and algorithms have begun to be explored to aid with structural variant identification. For example, DeepSVFilter is a deep learning based approach that has been used to attempt to replace the visual inspection step by encoding read depth, discordant read pairs and split reads from short-read sequencing into images. A convolutional neural network may be used to classify a structural variant as high-confidence or low-confidence. High-confidence structural variants are used as a true positive set and low-confidence structural variants are used as a true negative set. DeepSVFilter, however, relies on genome-wide structural variants to train the neural network (such that it cannot account for local sequencing trends) and only uses a single publicly available benchmark sample that has not undergone the same sequencing protocol as the subject sequence. DeepSVFilter also requires defining true positives and true negatives for training as opposed to training with a “normal” population. As another example, CHUNK is a random forest classifier that is trained on genome-wide structural variants that are high-confidence or low-confidence from publicly available cohorts. Both DeepSVFilter and CHUNK use supervised learning approaches, whereas an autoencoder is an unsupervised learning approach. Both DeepSVFilter and CHUNK are trained on structural variants from the entire genome and cannot account for local sequencing artifacts; however, an autoencoder specific for each structural variation can account for local sequencing artifacts. As yet another example, DeepVariant is a convolutional neural network that calls single nucleotide variants and small insertions/deletions from pileup images but cannot be used to call structural variants.

The embodiments, implementations, and/or examples, described herein may use an autoencoder to identify structural variants. As used herein, an “autoencoder” may refer to an artificial neural network used in unsupervised machine learning to encode (e.g., compress) and reconstruct input data. An autoencoder generally comprises an encoder, which encodes, maps, or embeds input data into one or more hidden layers of lower dimension (a latent presentation), and a decoder, which attempts to reconstruct the input data from the encoded data. Autoencoders are advantageous for analyzing noisy data as they can learn to distinguish between important and unimportant variations. The autoencoder may be used to substitute for the visual inspection step in a structural variation workflow. Alternatively, the autoencoder can be used upstream of the visual inspection step to filter out some false positive structural variants, thereby eliminating the need to inspect so many read depth plots. The autoencoder can be used downstream of the visual inspection step to filter out false positive structural variants.

### Example System Architecture

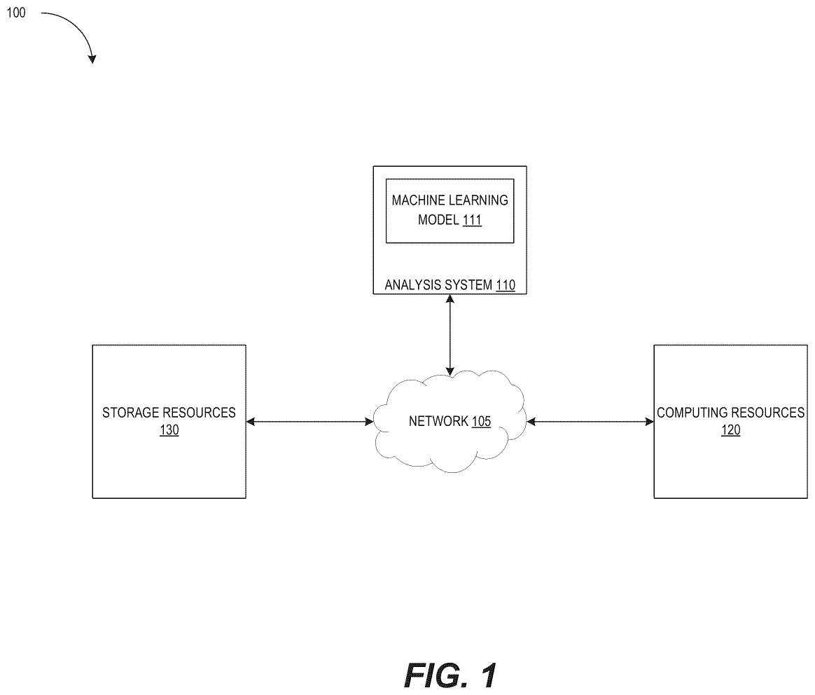

FIG. 1 is a block diagram that illustrates an example of system architecture 100, in accordance with some embodiments of the present disclosure. The system architecture 100 includes an analysis system 110, computing resources 120, and storage resources 130. One or more networks may interconnect the analysis system 110, the computing resources 120, and/or the storage resources 130. A network may be a public network (e.g., the internet), a private network (e.g., a local area network (LAN) or wide area network (WAN)), or a combination thereof. In one embodiment, network may include a wired or a wireless infrastructure, which may be provided by one or more wireless communications systems, such as a wireless fidelity (Wi-Fi) hotspot connected with the network, a cellular system, and/or a wireless carrier system that can be implemented using various data processing equipment, communication towers (e.g., cell towers), etc. The network may carry communications (e.g., data, message, packets, frames, etc.) between the analysis system 110, the computing resources 120 and/or the storage resources 130. Alternatively, the analysis system 110, computing resources 120, and/or the storage resources 130 may be part of a single computing device such that a network between any two or more of them is not required (not shown in FIG. 1). In such embodiments, the methods described herein may be performed entirely locally (e.g., on a single computer). The system architecture 100 may be implemented on a single computer or through the use of multiple devices connected via network 105.

The computing resource 120 may include computing devices which may include hardware such as processing devices (e.g., processors, central processing units (CPUs), processing cores, graphics processing units (GPUS)), memory (e.g., random access memory (RAM), storage devices (e.g., hard-disk drive (HDD), solid-state drive (SSD), etc.), and other hardware devices (e.g., sound card, video card, etc.). The computing devices may comprise any suitable type of computing device or machine that has a programmable processor including, for example, server computers, desktop computers, rackmount servers, etc. In some examples, the computing devices may include a single machine or may include multiple interconnected machines (e.g., multiple servers configured in a cluster, cloud computing resources, etc.).

The computing resources 120 may also include virtual environments. In one embodiment, a virtual environment may be a virtual machine (VM) that may execute on a hypervisor which executes on top of the OS for a computing device. The hypervisor may also be referred to as a virtual machine monitor (VMM). A VM may be a software implementation of a machine (e.g., a software implementation of a computing device) that includes its own operating system (referred to as a guest OS) and executes application programs, applications, software. The hypervisor may be a component of an OS for a computing device, may run on top of the OS for a computing device, or may run directly on host hardware without the use of an OS. The hypervisor may manage system resources, including access to hardware devices such as physical processing devices (e.g., processors, CPUs, etc.), physical memory (e.g., RAM), storage device (e.g., HDDs, SSDs), and/or other devices (e.g., sound cards, video cards, etc.). The hypervisor may also emulate the hardware (or other physical resources) which may be used by the VMs to execute software/applications. The hypervisor may present other software (i.e., “guest” software) the abstraction of one or more VMs that provide the same or different abstractions to various guest software (e.g., guest operating system, guest applications). A VM may execute guest software that uses an underlying emulation of the physical resources (e.g., virtual processors and guest memory).

In another embodiment, a virtual environment may be a container that may execute on a container engine which executes on top of the OS for a computing device, as discussed in more detail below. A container may be an isolated set of resources allocated to executing an application, software, and/or process independent from other applications, software, and/or processes. The host OS (e.g., an OS of the computing device) may use namespaces to isolate the resources of the containers from each other. A container may also be a virtualized object similar to virtual machines. However, a container may not implement separate guest OS (like a VM). The container may share the kernel, libraries, and binaries of the host OS with other containers that are executing on the computing device. The container engine may allow different containers to share the host OS (e.g., the OS kernel, binaries, libraries, etc.) of a computing device. The container engine may also facilitate interactions between the container and the resources of the computing device. The container engine may also be used to create, remove, and manage containers.

The storage resources 130 may include various different types of storage devices, such as hard disk drives (HDDs), solid state drives (SSD), hybrid drives, storage area networks, storage arrays, etc. The storage resources 130 may also include cloud storage resources or platforms which allow for dynamic scaling of storage space.

Although the computing resources 120 and the storage resources 130 are illustrated separate from the analysis system 110, one or more of the computing resources 120 and the storage resources 130 may be part of the analysis system 110 in other embodiments. For example, the analysis system 110 may include both the computing resources 120 and the storage resources 130. The analysis system 110 includes a machine learning model 111. The machine learning model 111 may be an autoencoder, as discussed in more detail below.

In one embodiment, the analysis system 110 may manage the allocation and/or use of computing resources 120 (e.g., computing clusters, server computers, VMs, containers, etc.). The computing resources 120 may be used for data transformation, feature extraction, development, generating training data, and testing of machine learning models, etc. The computing resources 120 may use various cloud service platforms (e.g., cloud computing resources). The analysis system 110 may also manage the allocation and/or use of storage resources 130. The storage resources 130 may store training data, machine learning models, and/or any other data used during the development and/or testing of machine learning models.

Some embodiments of the disclosure provide a system (e.g., analysis system 110) and method for identifying a structural variant. The method includes constructing read depth profiles from the sequencing data of a subject and reference samples. The read depth profiles (or simply “depth profiles”) may encompass a candidate structural variant region. The method may also include training an autoencoder based on read depth profiles from reference samples. The autoencoder may be used to reconstruct the read depth profile of the subject. The difference between the subject's reconstructed read depth profile and the subject's original read depth profile may be measured. A structural variant in the subject may be called based on the measured difference. As used herein, a structural variant may be “called” if it is identified as real or flagged as having a relatively increased likelihood of being real relative to an unevaluated SV candidate (e.g., selected for further analysis).

In one embodiment, the candidate structural variant is comprised of genome build, chromosome, start coordinate, and end coordinate. In other embodiments, the candidate structural variant is also comprised of the structural variation type (e.g., deletion, insertion, duplication, translocation, or inversion). A candidate structural variant region may be a region in a genome (e.g., as defined by one or more breakpoints, whereby a breakpoint can be described either by start and end positions/coordinates for a particular chromosome or by a single position/coordinate in a particular chromosome) for which at least a portion of the read depth profile for a subject genome in which the candidate structural variant is real would be expected to measurably deviate relative to the read depth profile expected for a reference genome as a result of the structural variation, when the subject genome is mapped to the reference genome. In some embodiments, the candidate structural variant region consists of only the region for which the presence of a real structural variant would be expected to alter the read depth profile or a portion of such a region. In some embodiments, the structural variant region may include regions spanning the breakpoints, where the presence of a real structural variant would be expected to deviate from normal at these breakpoints. In some embodiments, the candidate structural variant is a duplication for which the presence of a real duplication is expected to result in higher read depths compared to normal samples. In some embodiments, the candidate structural variant is a deletion for which the presence of a real deletion is expected to result in lower read depths compared to normal samples. In some embodiments, the candidate structural variant is an inversion or translocation for which the presence of a real structural variant is expected to result in lower read depths at the breakpoints compared to normal samples. In some embodiments, the candidate structural variant is identified from SV algorithms applied to the subject's genomic sequence (e.g., TIDDIT, CNVnator, Manta, Lumpy). In other embodiments, the candidate structural variant is obtained based on disease candidate structural variants from external verified sources such as various databases, published documents, or other data sources. In further embodiments, the candidate structural variant is obtained from databases. Examples of such databases are the OMIM database and/or the Clin Var database.

In one embodiment, a read depth profile as used herein may refer to the read depths of the candidate structural variant region derived from the sequencing data of a sample. The read depth profile can be the read depth per position (e.g., a 1000 base pair (bp) SV would have 1000 data points reflecting the reads at each position). In another embodiment, the read depth profile may be averaged across a window (e.g., a 1000 bp SV has 200 data points, each point represents the average read depth across a 5 bp window). In a further embodiment, the median instead of average can be used. In other embodiments, not all positions have to be sampled (e.g., sample every 1 kilobase (kb) position). This may save computational time for larger regions. Noise may be introduced into a read depth profile by any number of mechanisms, including, for example, by stochastic events due to sampling, GC bias, and/or the uneven distribution of variants across the genome, in addition to any copy number abnormality. The methods and/or systems described herein may be used to distinguish variation in read depth profiles resulting from noise from that resulting from structural variation.

In some embodiments, the profile includes flanking regions (e.g., 1 kb upstream and/or downstream of the SV). The size of the flanking region may be between about 5-25%, 5-20%, 5-15%, 5-10%, 10-25%, 10-20%, 10-15%, 15-25%, or 15-20% the size of the candidate structural variant. In some embodiments, the size of the flanking region may be about 10% the size of the candidate structural variant (e.g., 25 kb for a 250 kb structural variant). In other embodiments, the read depth is normalized to genome-wide coverage, autosomal chromosome coverage, or sex chromosome coverage. In further embodiments, the read depth profile is bounded by an upper limit to account for regions with higher read depth. In some embodiments, the read depth profile may span the breakpoints and flanking regions. These read depth profiles may be used, for example, for identifying balanced translocations and inversions. As used herein, “breakpoints” may refer to the location in a genome where a structural variant (e.g., the addition, deletion, or rearrangement of nucleotides) begins or ends relative to a reference genome.

The read depth profiles may be used to train the machine learning model 111. In one embodiment, the machine learning model 111 may be an autoencoder. In some embodiments, the autoencoder is a sparse autoencoder. In other embodiments, the autoencoder is a convolutional neural network. In other embodiments, the autoencoder is a regularized autoencoder. In other embodiments, the autoencoder is a variational autoencoder.

The machine learning model 111 (e.g., an autoencoder) can be trained on read depth profiles from training samples. In some embodiments, the training samples are control samples. In other embodiments, the training samples may correspond to reference samples that are obtained from a reference cohort. Examples of reference cohorts may include 1000 Genomes (The 1000 Genomes Consortium) and UK Biobank. In further embodiments, the training samples are samples that do not share the same phenotype or trait as the subject. In some embodiments, the subject can be a tumor and the training samples consist of non-tumor samples. In other embodiments, the machine learning model 111 (e.g., an autoencoder) is trained on read depth profiles that have the candidate structural variant such that the candidate structural variant can be identified from the other samples. In various embodiments, the machine learning model 111 may be trained on a plurality of training samples, such as, for example, at least about 10, 15, 20, 25, 30, 40, 50, 60, 70, 80, 90, 100, 200, 300, 400, 500, of 1,000 training samples.

The trained machine learning model 111 (e.g., an autoencoder) may take as input the subject's read depth profile and may output a reconstructed read depth profile. The autoencoder may be able to predict, based on its training, spikes and dips that would ordinarily occur in read depth from the sequencing absent the presence of a real structural variant. In one embodiment, the reconstruction error may refer to the difference between the subject's original read depth profile and reconstructed read depth. In some embodiments, the reconstruction error may be the mean-squared error between a reconstructed read depth profile and the original read depth profile. In some embodiments, the reconstruction error may be the Kullback-Leibler divergence of a reconstructed read depth profile from the original read depth profile.

In some embodiments, each trained machine learning model 111 (e.g., autoencoder) may be associated with a label. The label for a given machine learning model 111 may be indicative of the type of data (e.g., reference samples associated with a particular label) used to train the machine learning model 111. Each reference sample may be associated with one or more labels, which may correspond to a particular disease, population cohort (e.g., reference cohort), sample collection type, and/or the like associated with the reference sample. Each reference sample may automatically be associated with a label based on data associated with the reference sample (e.g., metadata and/or data labels assigned by a user). Additionally, or alternatively, each reference sample may be manually associated with a label by a user. For example, a reference sample may be assigned a label based on the characteristics associated with the reference sample such as an associated disease status (e.g., normal, diseased, breast cancer, autism, intellectual disability, ichthyosis, etc.), an associated reference population (e.g., European, Asian, and/or African descent), a sample collection type (e.g., embryo sample, fetus sample, tumor sample, blood sample, saliva sample, tissue sample, and/or the like). The machine learning model 111 may be trained using reference samples with particular labels (e.g., reference samples associated with the same label such that they share at least one characteristic). As such, the machine learning model 111 may be trained on population-specific data such that the parameters (e.g., weights) of the machine learning model 111 can effectively reconstruct read depth profiles for that particular population. The trained machine learning model 111 may output a reconstructed read depth profile for a subject read depth profile and the reconstructed read depth profile may also be associated with the corresponding label.

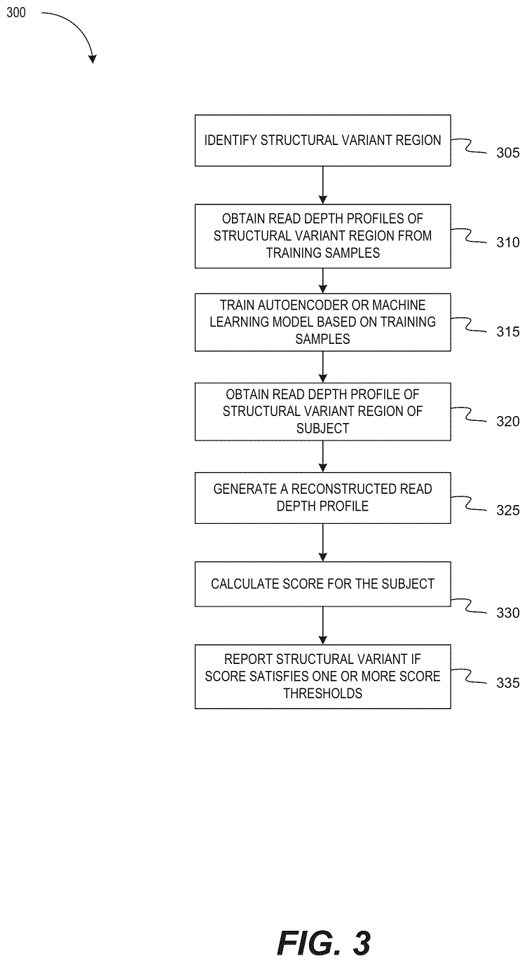

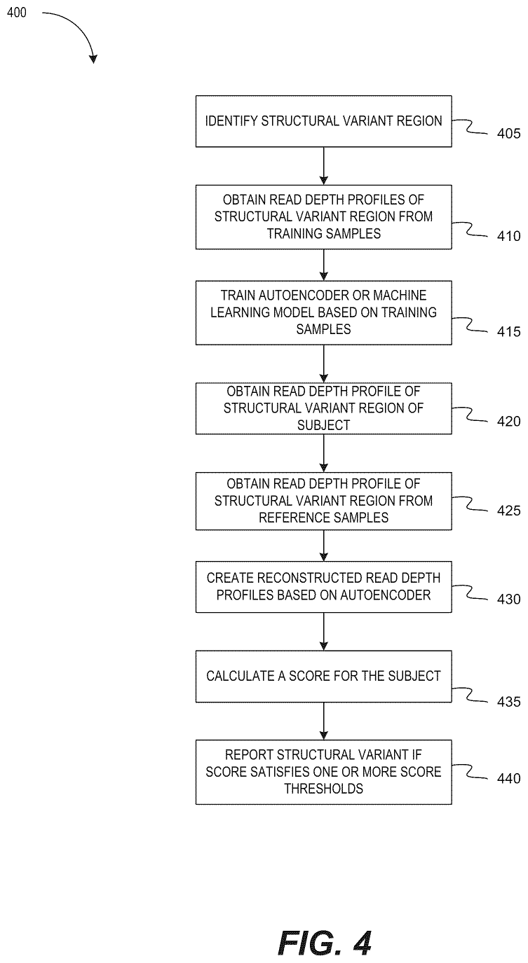

In one embodiment, the reconstruction error is used as a score to identify a real structural variant. In some embodiments, a SV is called, when the reconstruction error satisfies (e.g., exceeds) a score threshold (as depicted in FIG. 3 and exemplified in Examples 1 and 2). In other embodiments, the reconstruction errors are calculated for reference test samples and the subject's SV is identified based on the subject's reconstruction error compared to the reference samples' reconstruction errors (as depicted in FIG. 4 and exemplified in Example 3). In some embodiments, a z-score for the subject's reconstruction error is calculated from the distribution of reconstruction errors based on the reference samples. If the z-score of the subject satisfies (e.g., exceeds) a score threshold, the SV is called. In other embodiments, the subject's reconstruction error is ranked against reference test samples. If the subject's reconstruction error has a relatively high rank, then the SV is called.

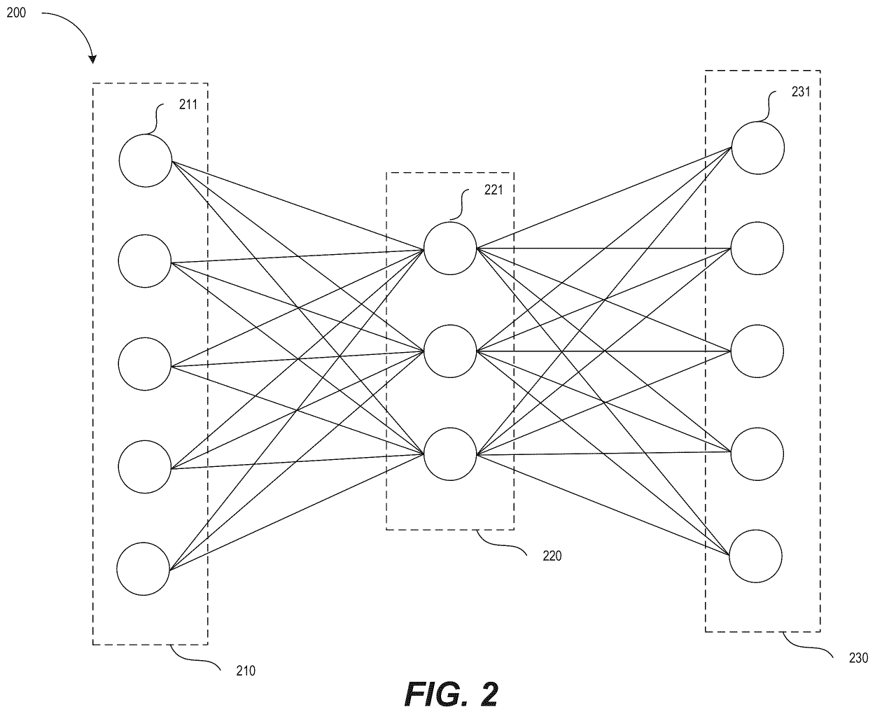

FIG. 2 is a diagram illustrating an example autoencoder 200, in accordance with one or more embodiments of the present disclosure. The autoencoder 200 may be an example of machine learning model 111 illustrated in FIG. 1. An autoencoder 200 may be used to model relationships between (e.g., complex) inputs and outputs or to find patterns in data, where the dependency between the inputs and the outputs may not be easily ascertained. The autoencoder 200 may also be a computing model that may be used to determine a feature in input data through various computations. For example, the autoencoder 200 may determine or learn data encodings in an unsupervised manner. The data encodings may be validated or verified by regenerating inputs from the data encodings.

The autoencoder 200 comprises an encoder layer 210, a hidden layer 220, and a decoder layer 230. The hidden layer 220 may also be referred to as a bottleneck. The encoder layer 210 may comprise nodes 211, the hidden layer 220 may comprise nodes 221, and the decoder layer 230 may comprise nodes 231. In some embodiments, one or more of the encoder layer 210, the hidden layer 220, and the decoder layer 230 may comprise multiple layers. For example, the encoder layer may comprise multiple layers of nodes 211. The autoencoder 200 may be a deep neural network (DNN) when or more of the encoder layer 210, the hidden layer 220, and the decoder layer 230 has multiple layers.

In one embodiment, the autoencoder 200 may be a feed forward neural network. A feed forward neural network may be a type of neural network where the connections between the nodes do not form a cycle. For example, the signals, messages, data, information etc., flow forward from the encoder layer 210, through hidden layer 220, to the decoder layer 230 (e.g., to the output nodes) of the autoencoder 200 from left to right. The signals, messages, data, information etc., may not go backwards through the autoencoder 200 (e.g., may not go from right to left).

Each of the nodes 211 in encoder layer 210 is connected to a node 221 in the hidden layer 220, as represented by the arrows/lines between the nodes 211 and 221. Each of the nodes 221 in encoder layer 220 is connected to a node 231 in the decoder layer 230, as represented by the arrows/lines between the nodes 221 and 231. Each connection may be associated with a weight or weight value (e.g., may have a weight). A weight or weight value may define coefficients applied to the computations. For example, the weights or weight values may be scaling factors between two or more nodes. Each node may represent a summation of its inputs, and the weight or weight value associated with a connection may represent a coefficient or a scaling factor multiplied to an output of a node in that connection. The weights between the nodes may be determined, calculated, generated, assigned, learned, etc., during a training process for the neural network. For example, backpropagation may be used to set the weights such that autoencoder 200 produces expected output values given corresponding values in labeled training data. Thus, the weights of the autoencoder 200 can be considered as an encoding of meaningful patterns in the data. The weights of the connections between the nodes may be modified by additional training.

In some embodiments, the autoencoder 200 may comprise nodes and/or layers that perform convolution operations. A convolution operation may refer to an operation that may merge two sets of information into an output. In other embodiment, the autoencoder 200 may comprise nodes and/or layers that perform pooling operations. A pooling operation may refer to down-sampling a feature map, to reduce the height and weight of the feature map, while retaining the same depth. In further embodiments, the autoencoder 200 may also comprise full connected layers.

Although autoencoder 200 is depicted with a particular number of nodes, layers, and connections, various neural network architectures/configurations may be used in other embodiments. Although the present disclosure may refer to autoencoders, other types of machine learning models, neural networks and/or deep neural networks may be used in other embodiments.

### Example Operations

FIG. 3 is a flow diagram illustrating an example process 300 for identifying structural variants, in accordance with one or more embodiments of the present disclosure. Process 300 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the process 300 may be performed by one or more computing devices, an analysis system (e.g., analysis system 100 illustrated in FIG. 1), a machine learning model (e.g., machine learning models 111 illustrated in FIG. 1), and/or an autoencoder (e.g., autoencoder 200 illustrated in FIG. 2).

With reference to FIG. 3, process 300 illustrates example functions used by various embodiments. Although specific function blocks (“blocks”) are disclosed in process 300, such blocks are examples. That is, embodiments are well suited to performing various other blocks or variations of the blocks recited in process 300. It is appreciated that the blocks in process 300 may be performed in an order different than presented, and that not all of the blocks in process 300 may be performed. In addition, additional other blocks (not illustrated in FIG. 3) may be inserted between the blocks illustrated in FIG. 3.

The process 300 begins at block 305 where the analysis system 110 is configured to identify a candidate structural variant region. The analysis system 110 may identify a candidate structural variant region based on one or more breakpoints associated with the structural variant region. As described above, a structural variant region may be a region in a genome (e.g., as defined by one or more breakpoints, whereby a breakpoint can be described either by start and end positions/coordinates for a particular chromosome or by a single position/coordinate in a particular chromosome) for which at least a portion of the read depth profile for a subject genome would be expected to measurably deviate relative to the read depth profile expected for a reference genome as a result of the structural variation, in the instance in which the structural variant is real. The analysis system 110 may identify the candidate structural variant region using one or more SV algorithms applied to the subject's genomic sequence (e.g., TIDDIT, CNVnator, Manta, Lumpy). Additionally, or alternatively, the analysis system 100 may identify the candidate structural variant region based on one or more configured breakpoint values. For example, one or more breakpoint values may be manually input and/or configured by an authorized user based on literature and/or database values.

At block 310, the analysis system 110 is configured to obtain read depth profiles of the structural variant region from training samples. In some embodiments, the read depth profiles from training samples may be stored in an associated memory and/or storage repository (e.g., storage resources 130, memory 604, static memory 606, etc.) and may be accessible by analysis system 110. Analysis system 110 may access and obtain the read depth profiles from the training samples in response to a request to train and/or retrain a machine learning model 111 and/or autoencoder 200. In some embodiments, the analysis system 110 may select a portion of available reference samples and designate these reference samples as training samples. In some embodiments, the remaining reference samples may be designated as test samples, as further discussed at block 425. For example, a repository may store 1000 reference samples and the analysis system 110 may designate 70 percent of the reference samples as training samples (e.g., 700 reference samples) and 30 percent of the reference samples as test samples (e.g., 300 reference samples). In some embodiments, the analysis system 110 may also designate a portion of the reference samples as validation samples. The analysis system 110 may receive this request from an authorized user and/or may be configured to periodically or semi-periodically retrain the machine learning model 111 and/or autoencoder 200. The read depth profiles accessed and obtained by the analysis system may pertain to the particular structural variant region identified by the analysis system 110 at block 305. It will be appreciated by one of skill in the art that block 310 may be not necessarily be required in an instance in which an autoencoder 200 and/or machine learning model 111 is already trained and the process may proceed directly to block 320.

At block 315, the analysis system 110 may be configured to train an autoencoder 200 and/or machine learning model 111 based on the obtained read depth profiles of the structural variant regions obtained from the training samples. For example, the analysis system 110 may provide the read depth profiles of the structural variant region from training samples as an input to the autoencoder 200 and/or machine learning model 111 to train the autoencoder 200 and/or machine learning model 111 (e.g., to set weights within the autoencoder and/or machine learning model). The optimized weights of the autoencoder 200 and/or machine learning model 111 may be stored in an associated memory and/or storage repository (e.g., storage resources 130, memory 604, static memory 606, etc.) such that these weights are accessible upon execution of the autoencoder 200 and/or machine learning model 111 in the future. It will be appreciated by one of skill in the art that block 315 may be not required in an instance in which an autoencoder 200 and/or machine learning model 111 is already trained and the process may in such instances proceed directly from block 305 or 310 to block 320.

At block 320, the analysis system 110 may be configured to obtain a read depth profile of a structural variant region of a subject. In some embodiments, the read depth profile of the structural variant region from the subject may be stored in an associated memory and/or storage repository (e.g., storage resources 130, memory 604, static memory 606, etc.) and may be accessible by analysis system 110. Analysis system 110 may access and obtain the read depth profile from the subject in response to a request to analyze the subject read depth profile, which may be provided by an authorized user. The read depth profile of the structural variant region from the subject obtained at block 320 may be referred to as the original read depth profile for the subject. In embodiments where this original read depth profile is not already stored in an accessible repository, the original read depth profile may be generated by the analysis system 110 or the analysis system 110 may prompt an out-of-band resource to generate the original read depth profile.

At block 325, the autoencoder 200 and/or machine learning model 111 may be configured to generate a reconstructed read depth profile for the subject. Once the analysis system 110 has obtained the read depth profile of the structural variant region for the subject (e.g., original read depth profile for the subject), the analysis system 110 may provide the read depth profile for the subject to the autoencoder 200 and/or machine learning model 111. The autoencoder 200 and/or machine learning model 111 may process the original read depth profile for the subject and generate a reconstructed read depth profile for the subject. The autoencoder 200 and/or machine learning model 111 may output the reconstructed read depth profile for the subject to the analysis system 110.

At block 330, the analysis system 110 may be configured to calculate a score for the subject. In some embodiments, the score is a reconstruction score (although the score may be a z-score in other embodiments, as described below in connection with FIG. 4). In particular, the analysis system 110 may be configured to calculate a reconstruction error for a subject based on differences between the reconstructed read depth profile for the subject and the original read depth profile for the subject that was obtained at block 320. The reconstruction error may be determined by calculating the mean squared error based on the original read depth profile and the reconstructed read depth profile for the subject. In particular, a difference between each data point in the original read depth profile and a corresponding data point in the reconstructed read depth profile for the subject may be calculated, squared and averaged and used to determine the reconstruction error for the subject.

At block 335, the analysis system 110 may be configured to determine whether the score determined for the subject satisfies one or more score thresholds. In an instance in which the analysis system 110 determines that the score (e.g., reconstruction error) for the subject satisfies one or more score thresholds, the analysis system 110 may report, call, or otherwise positively flag the structural variant. In some embodiments, the analysis system 110 may compare the score determined for the subject to one or more score thresholds. For example, a score threshold may be, for example, about or at least about 0.5, 0.6, 0.7, 0.8, 0.9, 1.0, 1.1, 1.2, 1.3, 1.4, 1.5, 1.6, 1.7, 1.8, 1.9, or 2.0. As another example, the analysis system 110 may determine whether the score (e.g., reconstruction error) for the subject is ranked in a predetermined top percentile of scores (e.g., reconstruction errors) calculated for a plurality of test samples and/or other reference samples. In an instance in which the score for the subject is ranked in a predetermined top percentile of scores, the analysis system may report the structural variant. As such, in an instance in which a score determined for a subject satisfies (e.g., exceeds) at least one of the score thresholds, the analysis system 110 may call, report, or flag the structural variant. Reporting the structural variant may be indicative that the candidate structural variant for the subject is determined to be real.

In some embodiments, once the structural variant is reported, called, or otherwise positively flagged, the analysis system 110 may output the original read depth profile and/or the reconstructed read depth profile of the subject to one or more users. In some embodiments, the analysis system 110 may also include an indication of the score (e.g., reconstruction error) determined for the subject such that a user may consider this value in a review of the read depth profiles.

FIG. 4 is a flow diagram illustrating an example process 400 for identifying structural variants, in accordance with one or more embodiments of the present disclosure. Process 400 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the process 400 may be performed by one or more of a computing device, an analysis system (e.g., analysis system 100 illustrated in FIG. 1), a machine learning model (e.g., machine learning models 111 illustrated in FIG. 1), and/or an autoencoder (e.g., autoencoder 200 illustrated in FIG. 2).

With reference to FIG. 4, process 400 illustrates example functions used by various embodiments. Although specific function blocks (“blocks”) are disclosed in process 400, such blocks are examples. That is, embodiments are well suited to performing various other blocks or variations of the blocks recited in process 400. It is appreciated that the blocks in process 400 may be performed in an order different than presented, and that not all of the blocks in process 400 may be performed. In addition, additional other blocks (not illustrated in FIG. 4) may be inserted between the blocks illustrated in FIG. 4.

The process 400 begins at block 405 where the analysis system 110 is configured to identify a candidate structural variant region. The analysis system 110 may identify a candidate structural variant region based on one or more breakpoints associated with the structural variant region. As described above, a structural variant region may be a region in a genome (e.g., as defined by one or more breakpoints, whereby a breakpoint can be described either by start and end positions/coordinates for a particular chromosome or by a single position/coordinate in a particular chromosome) for which at least a portion of the read depth profile for a subject genome in which the candidate structural variant is real would be expected to measurably deviate relative to the read depth profile expected for a reference genome as a result of the structural variation, when the subject genome is mapped to the reference genome. The analysis system 110 may identify the candidate structural variant region using one or more SV algorithms applied to the subject's genomic sequence (e.g., TIDDIT, CNVnator, Manta, Lumpy). Additionally, or alternatively, the analysis system 100 may identify the candidate structural variant region based on one or more configured breakpoint parameters. For example, one or more breakpoint parameters may be manually input and/or configured by an authorized user based on literature and/or database values.

At block 410, the analysis system 110 is configured to obtain read depth profiles of the structural variant region from training samples. In some embodiments, the read depth profiles from training samples may be stored in an associated memory and/or storage repository (e.g., storage resources 130, memory 604, static memory 606, etc.) and may be accessible by analysis system 110. Analysis system 110 may access and obtain the read depth profiles from the training samples in response to a request to train and/or retrain a machine learning model 111 and/or autoencoder 200. In some embodiments, the analysis system 110 may select a portion of available reference samples and designate these reference samples as training samples. In some embodiments, the remaining reference samples may be designated as test samples, as further discussed at block 425. For example, a repository may store 1000 reference samples and the analysis system 110 may designate 70 percent of the reference samples as training samples (e.g., 700 reference samples) and 30 percent of the reference samples as test samples (e.g., 300 reference samples). In some embodiments, the analysis system 110 may also designate a portion of the reference samples as validation samples. The analysis system 110 may receive this request from an authorized user and/or may be configured to periodically or semi-periodically retrain the machine learning model 111 and/or autoencoder 200. The read depth profiles accessed and obtained by the analysis system may pertain to the particular structural variant region identified by the analysis system 110 at block 405. It will be appreciated by one of skill in the art that block 410 may be not necessarily be required in an instance in which an autoencoder 200 and/or machine learning model 111 is already trained and the process may proceed directly to block 420.

At block 415, the analysis system 110 may be configured to train an autoencoder 200 and/or machine learning model 111 based on the obtained read depth profiles of the structural variant regions obtained from the training samples. For example, the analysis system 110 may provide the read depth profiles of the structural variant region from training samples as an input to the autoencoder 200 and/or machine learning model 111 to train the autoencoder 200 and/or machine learning model 111 (e.g., to set weights within the autoencoder and/or machine learning model). The optimized weights of the autoencoder 200 and/or machine learning model 111 may be stored in an associated memory and/or storage repository (e.g., storage resources 130, memory 604, static memory 606, etc.) such that these weights are accessible upon execution of the autoencoder 200 and/or machine learning model 111 in the future. It will be appreciated by one of skill in the art that block 415 may be not required in an instance in which the autoencoder 200 and/or machine learning model 111 is already trained and the process may proceed directly to block 420.

At block 420, the analysis system 110 may be configured to obtain a read depth profile of a structural variant region of a subject. In some embodiments, the read depth profile of the structural variant region from the subject may be stored in an associated memory and/or storage repository (e.g., storage resources 130, memory 604, static memory 606, etc.) and may be accessible by analysis system 110. Analysis system 110 may access and obtain the read depth profile from the subject in response to a request to analyze the subject read depth profile, which may be provided by an authorized user. The read depth profile of the structural variant region from the subject obtained at block 420 may be referred to as the original read depth profile for the subject. In embodiments where this original read depth profile is not already stored in an accessible repository, the original read depth profile may be generated by the analysis system 110 or the analysis system 110 may prompt an out-of-band resource to generate the original read depth profile.

At block 425, the analysis system 110 may be configured to obtain one or more read depth profiles of a structural variant region from one or more reference samples, which may be test samples. In some embodiments, the one or more read depth profiles of the structural variant region from reference samples may be stored in an associated memory and/or storage repository (e.g., storage resources 130, memory 604, static memory 606, etc.) and may be accessible by analysis system 110. Analysis system 110 may access and obtain the one or more read depth profiles from the reference samples in response to the request to analyze the subject read depth profile as described above at block 420. In some embodiments, the analysis system 110 may access and obtain the original read depth profile of the subject and the one or more read depth profiles of the structural variant region from the reference samples simultaneously. A read depth profile of the structural variant region from a reference sample obtained at block 425 may be referred to as the original read depth profile for the particular reference sample. It will be appreciated that in some embodiments where these original read depth profiles have previously been obtained by the analysis system 110 at block 410, the original read depth profiles may be stored locally by the analysis system 110 for quicker retrieval in block 425.

In various embodiments, a plurality of reference samples is obtained, such as, for example, at least about 10, 15, 20, 25, 30, 40, 50, 60, 70, 80, 90, 100, 200, 300, 400, 500, or 1,000 reference samples. The number may be no less than a minimum number of reference samples needed to maintain a desired margin of error. The reference samples in block 425 are preferably different reference samples from those used to train the autoencoder (e.g., the training samples).

At block 430, the autoencoder 200 and/or machine learning model 111 may be configured to generate a reconstructed read depth profile for the subject and corresponding reconstructed read depth profiles for the one or more reference samples. To do this, the analysis may originally obtain the original read depth profiles for the subject and the one or more reference samples. Once the analysis system 110 has obtained the read depth profile of the structural variant region for the subject (e.g., original read depth profile for the subject obtained at block 420) and the one or more the read depth profiles of the structural variant region for the one or more reference samples (e.g., original read depth profile for the one or more reference samples obtained at block 425), the analysis system 110 may provide these original read depth profiles to the autoencoder 200 and/or machine learning model 111. The autoencoder 200 and/or machine learning model 111 may process the original read depth profile for the subject and the one or more original read depth profiles for the one or more reference samples and generate a reconstructed read depth profile for each. The autoencoder 200 and/or machine learning model 111 may output the reconstructed read depth profile for the subject and the reconstructed read depth profiles for the one or more reference samples to the analysis system 110.

At block 435, the analysis system 110 may be configured to calculate a score for the subject. In some embodiments, the score is a z-score (although as described previously in connection with FIG. 3, other scores are contemplated in other embodiments). In particular, the analysis system 110 may be configured to calculate a reconstruction error for the subject and for each reference sample based on differences between each reconstructed read depth profile and a corresponding original read depth profile (obtained at block 420 for the subject and obtained at block 425 for each reference sample). The reconstruction error may be determined by calculating the mean squared error based on the original read depth profile and the reconstructed read depth profile. In particular, a difference between each data point in the original read depth profile and a corresponding data point in the reconstructed read depth profile for the subject may be calculated, squared and averaged and used to determine the reconstruction error for the subject. A z-score for the subject may then be calculated for the subject. The z-score may be calculated as a measure of the subject's reconstruction error relative to the reconstruction errors generated for the one or more reference samples, and is calculated from the distribution of reconstruction errors generated at block 430 based on the read depth profiles for the structural variant region for the subject and the one or more reference samples.

At block 440, the analysis system 110 may be configured to determine whether the score determined for the subject satisfies one or more score thresholds. In an instance in which the analysis system 110 determines that the score (e.g., z-score) for the subject satisfies one or more score thresholds, the analysis system 110 may report, call, or otherwise positively flag the structural variant. In some embodiments, the analysis system may compare the score determined for the subject to one or more score thresholds. For example, a score threshold may be, for example, about or at least about 1.65, 1.96, 2.0, 2.58 or 3.0. As another example, the analysis system 110 may determine whether the score (e.g., z-score) for the subject is ranked in a predetermined top percentile of scores (e.g., z-scores) calculated for a plurality of test samples and/or other reference samples. In an instance in which the score for the subject is ranked in a predetermined top percentile of scores, the analysis system may report the structural variant. As such, in an instance in which a score determined for a subject satisfies (e.g., exceeds) at least one of the score thresholds, the analysis system 110 may call, report, or flag the structural variant. Reporting the structural variant may be indicative that the candidate structural variant for the subject is determined to be real.

In some embodiments, once the structural variant is reported, called, or otherwise positively flagged, the analysis system 110 may output the original read depth profile and/or the reconstructed read depth profile of the subject to one or more users. In some embodiments, the analysis system 110 may also include an indication of the score (e.g., z-score) determined for the subject such that a user may consider this value in a review of the read depth profiles. In some embodiments, the analysis system 110 may also output one or more of the original read depth profiles and/or reconstructed profiles for one or more reference samples.

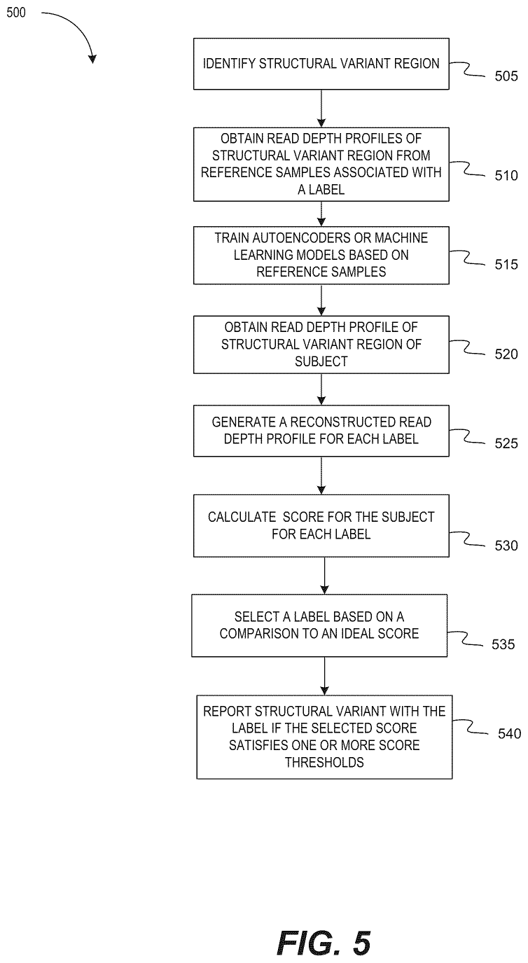

FIG. 5 is a flow diagram illustrating an example process 500 for identifying structural variants associated with a particular label, in accordance with one or more embodiments of the present disclosure. Process 500 may be performed by processing logic that may comprise hardware (e.g., circuitry, dedicated logic, programmable logic, a processor, a processing device, a central processing unit (CPU), a system-on-chip (SoC), etc.), software (e.g., instructions running/executing on a processing device), firmware (e.g., microcode), or a combination thereof. In some embodiments, the process 500 may be performed by one or more of a computing device, an analysis system (e.g., analysis system 110 illustrated in FIG. 1), a machine learning model (e.g., machine learning models 111 illustrated in FIG. 1), and/or an autoencoder (e.g., autoencoder 200 illustrated in FIG. 2).

With reference to FIG. 5, process 500 illustrates example functions used by various embodiments. Although specific function blocks (“blocks”) are disclosed in process 500, such blocks are examples. That is, embodiments are well suited to performing various other blocks or variations of the blocks recited in process 500. It is appreciated that the blocks in process 500 may be performed in an order different than presented, and that not all of the blocks in process 500 may be performed. In addition, additional other blocks (not illustrated in FIG. 5) may be inserted between the blocks illustrated in FIG. 5.

The process 500 begins at block 505 where the analysis system 110 is configured to identify a candidate structural variant region. The analysis system 110 may identify a candidate structural variant region based on one or more breakpoints associated with the structural variant region. As described above, a structural variant region may be a region in a genome (e.g., as defined by one or more breakpoints, whereby a breakpoint can be described either by start and end positions/coordinates for a particular chromosome or by a single position/coordinate in a particular chromosome) for which at least a portion of the read depth profile for a subject genome in which the candidate structural variant is real would be expected to measurably deviate relative to the read depth profile expected for a reference genome as a result of the structural variation, when the subject genome is mapped to the reference genome. The analysis system 110 may identify the candidate structural variant region using one or more SV algorithms applied to the subject's genomic sequence (e.g., TIDDIT, CNVnator, Manta, Lumpy). Additionally or alternatively, the analysis system 100 may identify the candidate structural variant region based on one or more configured breakpoint parameters. For example, one or more breakpoint parameters may be manually input and/or configured by an authorized user based on literature and/or database values.

At block 510, the analysis system 110 is configured to obtain read depth profiles of the structural variant region from reference samples associated with the same label. In some embodiments, each reference sample may be associated with one or more labels. As described above, a reference sample may be assigned a label based on the characteristics associated with the reference sample such as a disease status (e.g., normal, diseased, breast cancer, autism, intellectual disability, ichthyosis, etc.), membership in a specific population (e.g., European, Asian, and/or African descent), a sample collection type (e.g., embryo sample, fetus sample, tumor sample, blood sample, saliva sample, tissue sample, and/or the like). The analysis system 110 may be configured to obtain read depth profiles of the structural variant region from reference samples associated with one or more particular labels. In particular, the analysis system 110 may be configured to obtain and/or access read depth profiles of reference samples associated with labels that correspond to a machine learning model and/or autoencoder to be trained for the corresponding label.

In some embodiments, the read depth profiles from reference samples may be stored in an associated memory and/or storage repository (e.g., storage resources 130, memory 604, static memory 606, etc.) and may be accessible by analysis system 110. Analysis system 110 may access and obtain the read depth profiles from the reference samples in response to a request to train and/or retrain a machine learning model 111 and/or autoencoder 200. In some embodiments, the analysis system 110 may select a portion of available reference samples and designate these reference samples as training samples. In some embodiments, the remaining reference samples may be designated as test samples. For example, a repository may store 1000 reference samples and the analysis system 110 may designate 70 percent of the reference samples as training samples (e.g., 700 reference samples) and 30 percent of the reference samples as test samples (e.g., 300 reference samples). In some embodiments, the analysis system 110 may also designate a portion of the reference samples as validation reference samples. The analysis system 110 may receive this request from an authorized user and/or may be configured to periodically or semi-periodically retrain the machine learning model 111 and/or autoencoder 200. The read depth profiles accessed and obtained by the analysis system may pertain to the particular structural variant region identified by the analysis system 110 at block 505. It will be appreciated by one of skill in the art that block 510 may not necessarily be required in an instance in which an autoencoder 200 and/or machine learning model 111 is already trained and the process may proceed directly to block 520.

At block 515, the analysis system 110 may be configured to train multiple autoencoders 200 and/or machine learning models 111 associated with corresponding labels and based on relevant subsets of the obtained read depth profiles of the structural variant regions obtained from the reference samples. For example, the analysis system 110 may provide the read depth profiles of the structural variant region from reference samples associated with one or more labels as an input to the autoencoder 200 to train the autoencoder 200 (e.g., to set weights within the autoencoder). The optimized weights of the autoencoder 200 may be stored in an associated memory and/or storage repository (e.g., storage resources 130, memory 604, static memory 606, etc.) such that these weights are accessible upon execution of the autoencoder 200 in the future. As such, the autoencoder 200 and/or machine learning model 111 may be trained for a particular label and is therefore associated with the particular label.

In addition, a secondary autoencoder and/or secondary machine learning model may be trained for and associated with a secondary label that is different from the first label. In such an instance, the secondary autoencoder and/or machine learning model may be trained based on the obtained read depth profiles of the structural variant regions obtained from one or more secondary reference samples that are associated with the secondary label. For example, the analysis system 110 may provide the read depth profiles of the structural variant region from reference samples associated with the secondary label as an input to the secondary autoencoder to train the secondary autoencoder (e.g., to set weights within the autoencoder). The optimized weights of the secondary autoencoder may be stored in an associated memory and/or storage repository (e.g., storage resources 130, memory 604, static memory 606, etc.) such that these weights are accessible upon execution of the secondary autoencoder in the future. As such, the secondary autoencoder and/or secondary machine learning model may be trained for a secondary label and is therefore associated with the secondary label. It will be appreciated by one of skill in the art that block 515 may be not required in an instance in which an autoencoder 200 and/or machine learning model 111 is already trained and the process may proceed directly to block 520.

At block 520, the analysis system 110 may be configured to obtain a read depth profile of a structural variant region of a subject. In some embodiments, the read depth profile of the structural variant region from the subject may be stored in an associated memory and/or storage repository (e.g., storage resources 130, memory 604, static memory 606, etc.) and may be accessible by analysis system 110. Analysis system 110 may access and obtain the read depth profile from the subject in response to a request to analyze the subject read depth profile, which may be provided by an authorized user. The read depth profile of the structural variant region from the subject obtained at block 320 may be referred to as the original read depth profile for the subject. In embodiments where this original read depth profile is not already stored in an accessible repository, the original read depth profile may be generated by the analysis system 110 or the analysis system 110 may prompt an out-of-band resource to generate the original read depth profile.

At block 525, the autoencoder 200 and/or machine learning model 111 may be configured to generate one or more reconstructed read depth profiles for the subject for each label. Once the analysis system 110 has obtained the original read depth profile of the structural variant region for the subject (e.g., original read depth profile for the subject), the analysis system 110 may provide the read depth profile for the subject to each autoencoder 200 and/or machine learning model 111 associated with a corresponding label. Each autoencoder 200 and/or machine learning model 111 may process the original read depth profile for the subject and generate a reconstructed read depth profile for the subject for its corresponding label. Each autoencoder 200 and/or machine learning model 111 may output its reconstructed read depth profile for the subject to the analysis system 110. Each reconstructed read depth profile for the subject may therefore be associated with the label for which its corresponding autoencoder 110 and/or machine learning model 111 was trained.

For example, the analysis system 110 may use an autoencoder 200 and/or machine learning model 111 associated with a disease label to generate a reconstructed read depth profile for the subject associated with a disease label and a secondary autoencoder and/or secondary machine learning model associated with a normal label to generate a reconstructed read depth profile for the subject associated with normal label.

At block 530, the analysis system 110 may be configured to calculate a score for the subject for each label. In some embodiments, a score is a reconstruction score and/or a z-score as described above in block 330 and block 435, respectively. In particular, the analysis system 110 may be configured to calculate a score for the subject for each particular label based on the original read depth profile of the subject and the reconstructed read depth profile of the subject corresponding to that particular label and autoencoder. By way of continuing example, a first score may be calculated for a subject using the original read depth profile of the subject and a reconstructed read depth profile for the subject from the autoencoder trained on samples with disease labels. Similarly, a second score may be calculated for a subject using the original read depth profile of the subject and a reconstructed read depth profile from the autoencoder trained on samples labelled as normal.

At block 535, the analysis system 110 may be configured to select a label based on a comparison of each calculated score to an ideal score. An ideal score may be a pre-configured score that is set by an authorized user. For example, the ideal score may be a value of zero, which may indicate that no error exists between an original read depth profile and a reconstructed read depth profile. The analysis system 110 may compare each score, as calculated at block 530, to the ideal score and select the label based on this comparison. In some embodiments, the analysis system 110 may select the label that corresponds to the score which is closest to the ideal score. For example, the analysis system 110 may select the label that corresponds to the score which is closest to zero. In some embodiments, the analysis system 110 may take the absolute value of the scores when comparing them to the ideal score. Additionally, each score may be associated with a label that corresponds to the autoencoder that generated the corresponding reconstructed read depth profile.

By way of example, a first autoencoder associated with a first label may generate a first reconstructed read depth profile. A first score may be calculated based on a difference or deviation between the first reconstructed read depth profile and the original read depth profile. Additionally, a secondary autoencoder associated with a second label that is different than the first label, may generate a second reconstructed read depth profile. A secondary score may be calculated based on a difference or deviation between the secondary reconstructed read depth profile and the original read depth profile. The analysis system 110 may then select either the first score or the secondary score based on a comparison of the respective scores to the ideal score. As a particular example, the ideal score may be zero and the analysis system 110 may select the score closest to zero.

At block 540, the analysis system 110 may be configured to determine whether the selected score determined for the subject satisfies one or more score thresholds. In an instance in which the analysis system 110 determines that the selected score (e.g., reconstruction score or z-score) for the subject satisfies one or more score thresholds, the analysis system 110 may report, call, or otherwise positively flag the structural variant. Additionally, in an instance in which the analysis system 110 has selected the score in the manner described above in block 535, the analysis system may further report the structural variant with the label that corresponds to the selected score. In some embodiments, the analysis system may compare the selected score determined for the subject to one or more score thresholds and/or may determine whether the selected score (e.g., reconstruction score or z-score) for the subject is ranked in a predetermined top percentile of scores calculated for a plurality of test samples and/or other reference samples, as described above.

By way of continued example, the analysis system 110 may select the secondary score, which may be associated with the second label. As such, the analysis system 110 may determine whether the secondary score satisfies the one or more score thresholds. In an instance in which the secondary score does satisfy the one or more score thresholds, the analysis system 110 may report the structural variant as real and with the second label.

In some embodiments, once the structural variant is reported, called, or otherwise positively flagged, the analysis system 110 may output the original read depth profile and/or the reconstructed read depth profile of the subject that is associated with the selected score to one or more users. The analysis system 110 may further include the label associated with the selected score such that the users may be made aware of a label determined to best correspond to the read depth profile of the subject. In some embodiments, the analysis system 110 may also include an indication of the selected score and optionally, the other one or more calculated scores that were not selected. In some embodiments, the analysis system 110 may also output one or more of the original read depth profiles and/or reconstructed profiles for one or more reference samples.

Advantageously, since the methods described herein train the autoencoders on read depth profiles generated from sequencing data for reference samples, the autoencoders can account for variation in read depth caused by upstreaming sequencing methods, including both laboratory techniques (e.g., type of sequencing) and bioinformatics (e.g., genome mapping strategies). Also, because the autoencoder can be trained for each specific candidate structural variant (e.g., by defining an isolated candidate structural variant region) the autoencoder may be able to more accurately identify all individual structural variants, as compared, for example, to applying approaches that analyze a whole genome for trying to identify or predict structural variants. Surprisingly, the use of simple neural networks and raw sequencing data (e.g., as opposed to analysis of images or plots) has been shown to provide better performance. Without being bound by theory, simpler networks and/or the use of raw data may advantageously prevent overfitting of the input data.

### Example Implementing Apparatuses

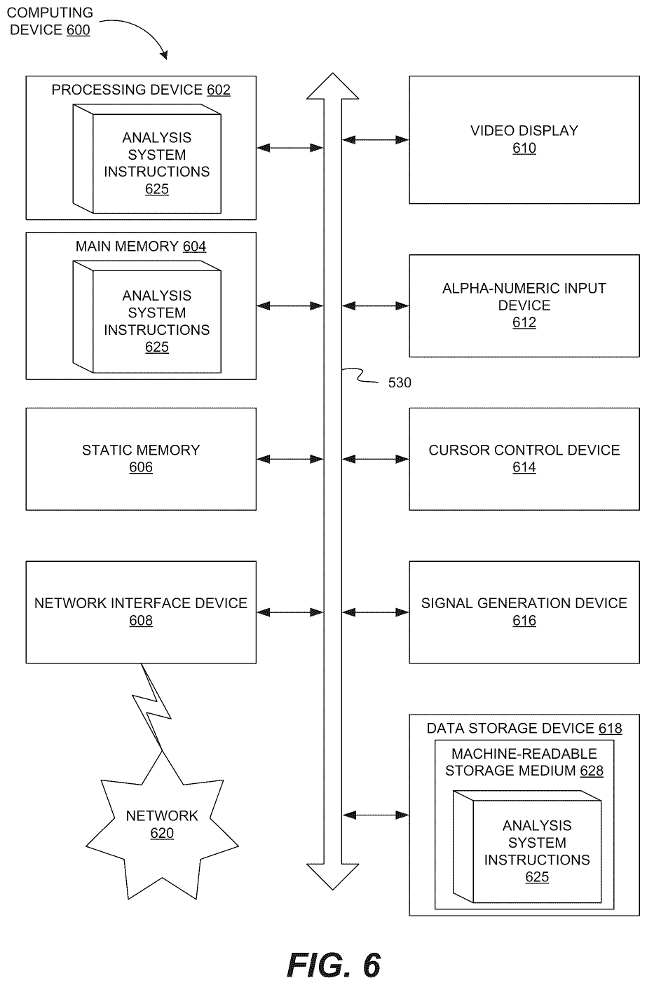

FIG. 6 is a block diagram of an example computing device 600, in accordance with some embodiments. Computing device 600 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” shall also be taken to include any collection of computing devices that individually or jointly execute a set (or multiple sets) of instructions to perform the methods discussed herein.

The example computing device 600 may comprise a processing device 602 (e.g., a general purpose processor, a programmable logic device (PLD), etc.), a main memory 604 (e.g., synchronous dynamic random access memory (DRAM), read-only memory (ROM)) (not shown), a static memory 606 (e.g., flash memory), and a data storage device 618), which may communicate with each other via a bus 630.

Processing device 602 may be provided by one or more general-purpose processing devices such as a microprocessor, central processing unit, or the like. In an illustrative example, processing device 602 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 602 may also comprise one or more special-purpose processing devices such as an application specific integrated circuit (ASIC), a field programmable gate array (FPGA), a digital signal processor (DSP), network processor, or the like. The processing device 602 may be configured to execute the operations described herein, in accordance with one or more aspects of the present disclosure, for performing the operations and steps discussed herein.

Computing device 600 may further comprise a network interface device 608 which may communicate with a network 620. The computing device 600 also may comprise a video display unit 610 (e.g., a liquid crystal display (LCD) or a cathode ray tube (CRT)), an alphanumeric input device 612 (e.g., a keyboard), a cursor control device 614 (e.g., a mouse) and an acoustic signal generation device 616 (e.g., a speaker). In one embodiment, video display unit 610, alphanumeric input device 612, and cursor control device 614 may be combined into a single component or device (e.g., an LCD touch screen).

Data storage device 618 may comprise a computer-readable storage medium 628 on which may be stored one or more sets of analysis system instructions 625, e.g., instructions for carrying out the operations described herein, in accordance with one or more aspects of the present disclosure. Analysis system instructions 625 may also reside, completely or at least partially, within main memory 604 and/or within processing device 602 during execution thereof by computing device 600, main memory 604 and processing device 602 also constituting computer-readable media. The analysis system instructions 625 may further be transmitted or received over a network 620 via network interface device 608.

While computer-readable storage medium 628 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” shall also be taken to include any medium that is capable of storing, encoding or carrying a set of instructions for execution by the machine and that cause the machine to perform the methods described herein. The term “computer-readable storage medium” shall accordingly be taken to include, but not be limited to, solid-state memories, optical media and magnetic media.

### Applications of Structural Variant Determinations

Various potential applications of evaluating candidate structural variants are possible. Applications, including various treatments and/or diagnostic testing, may include any suitable application of ploidy status determination, such as discussed in PCT App. No. PCT/US2021/057400 to Kumar et al., filed on Oct. 29, 2021, which is herein incorporated by reference in its entirety. Described herein are several specific, but non-limiting, examples of how such determinations can be used to drive subsequent decisions and/or further analysis or treatments.

### Genetically Profiling Tumors Having Chromosomal Instability

Genomic instability of tumor cells is often associated with poor patient outcome and resistance to targeted cancer therapies. The accumulation of genetic and epigenetic lesions in response to environmental exposures to carcinogens and/or random cellular events often results in the inactivation of tumor suppressor genes that play critical roles in the maintenance of cell cycle, DNA replication and DNA repair. Loss or inhibition of cellular DNA repair mechanisms often results in an increased mutation burden and genomic instability. Structural variants, such as CNVs, are prevalent across many types of cancer types and may cause the gain of oncogenes and/or loss of tumor suppressors associated with disease progression and therapeutic response or resistance. Genomic instability is associated with sub-clonal heterogeneity and is frequently observed in solid tumors between different lesions, within the same tumor, and even within the same solid biopsy site. Such tumor cell heterogeneity can complicate therapeutic intervention designed around single molecular targets. Genome-wide CNV profiles can be used to characterize genomic instability. However, assessment of genomic instability in bulk tumor or biopsy can be complicated due to sample availability as well as noise stemming from surrounding tissue contamination or tumor heterogeneity. Tumors associated with increased genomic instability have been shown to respond to specific types of therapies, including, for example, platinum-based chemotherapy and PARP inhibitors.

Treatment with poly ADP ribose polymerases (PARPs) or platinum-based chemotherapeutic (antineoplastic drugs, informally called “platins”) may be selected as treatments for cancers exhibiting chromosomal instability, as described, for example, in WO/2022/094310 to Kumar et al. Other forms of treatment that are appropriate for cancers exhibiting chromosomal instability are understood in the art. Accordingly, the methods described herein may relate to identifying genetic signatures in subjects having cancer that are indicative of chromosomal instability and, therefore, suitable for classes of therapeutics targeting genetic mechanisms (e.g., inhibiting the repair of DNA so that the damaged DNA may be more effectively targeted). These therapeutics may be agnostic to the specific type of cancer. Accordingly, the methods described herein may be performed on subjects diagnosed as having or suspected of having cancer prior to or concurrently with specific cancer diagnoses and/or tissue biopsies. The genetic analysis described herein may be performed concurrently with other routine analyses and/or cancer diagnoses or assessment based on the same or different biological samples collected at the same time.

According to specific embodiments, a read depth profile may be generated from a sample of genetic material collected from the subject. The profile may be generated from cell-free DNA which comprises or is suspected of comprising ctDNA. The profile may be generated from cellular DNA, such as tumor tissue. The cellular DNA may be obtained from blood cells (e.g., white blood cells). A candidate structural variant may be evaluated from the profile according to the systems and methods described herein to determine if the structural variant is real. The determination may be made with respect to a reference genetic code (e.g., a normal cell genetic code), as described elsewhere herein. The detection of one or more chromosomal segments exhibiting structural variation may be used to identify one or more regions of the genome displaying chromosomal instability. The identification of such regions may be used to indicate the presence of tumors that are susceptible to treatment with therapeutics that exploit chromosomal instability, such as treatment with PARP inhibitors and/or platinum-based chemotherapeutics. In some embodiments, the chromosomal instability determination is used to treat the subject (e.g., by administering the treatment in vivo). In some embodiments, the chromosomal instability determination is used to treat one or more cells in vitro. The one or more cells may comprise cancer cells. The cells may have been cultured from a subject having or suspected of having cancer (e.g., grown from a tumor biopsy). The cells may comprise cells from an cancer cell line (e.g., artificially induced to replicate a cancer). The cells may comprise a mixture of normal cells and cancerous cells.

### De Novo or Inherited CNV Detection

The methods and/or systems described herein may be used to call structural variations, such as CNVs, in a subject. A read depth profile may be used to call a structural variant, as described elsewhere herein. The methods described herein may be used to detect inherited structural variations (i.e. a structural variation at one or more loci of one of a subject's chromosomes, which was inherited from a parent) or de novo structural variations. If the structural variant is present in the genetic code of either of the parents, then the variant can be determined to be inherited. If the structural variant is not present in the genetic code of either of the parents, then the variant can be called as a de novo variation.

In some embodiments, a parent from whom a structural variant was inherited is determined. Additional sequencing may be performed on one (the originating parent) or both of the parents to confirm the determination. For example, whole genome sequencing (e.g., shotgun sequencing) may be performed on the parent(s), which may allow confirmation of the corresponding copy number in the originating parent. Calling of structural variants for an embryo or fetus (including calls of de novo changes) may generally be performed as described elsewhere herein (e.g., for a born child or adult individual).

Examples of specific associations between structural variants (e.g., CNVs or whole chromosomal abnormalities) and disease are well known in the art. In some implementations, the evaluation of structural variation may be used to inform decisions on IVF. The methods described herein may be performed on a single embryo or on a plurality of embryos (e.g., a plurality of embryo candidates for implantation). The evaluation of structural variation may be used to select one or more embryo's for implantation and/or to select one or more embryo's for discarding/disposal. The evaluation of structural variation may be used to select one or more embryo's for freezing (either in the case that the embryo is selected for possible future implantation or in the case that the embryo is not a primary candidate for implantation but it is not desired to be disposed of). For example, a determination of risk of disease may be made for an embryo at least in part based on the calling of a structural variant for a chromosome or chromosomal segment (e.g., the identification of a CNV, particularly one having a known association with a disease). In some implementations, an embryo with no identified structural variants (e.g., CNVs) may be selected for implantation or freezing. In some implementations, the embryos may be ranked based entirely or at least in part on the evaluation of structural variation (e.g., by the number of CNVs and/or the presence of particular CNVs). The evaluation of structural variation according to the methods described herein may be used independently or in combination with existing methods of preimplantation genetic testing (PGT), as is well known in the art.

According to some implementations, the evaluation of structural variation may be used to inform decisions on pregnancy, particularly where the subject is a fetus. For example, the decision whether to continue or terminate a pregnancy may be based on the evaluation of structural variation (e.g., the calling of a structural variant) in the same manner as decisions are made regarding IVF, as described elsewhere herein. The evaluation of structural variation according to the methods described herein may be used independently or in combination with existing methods of prenatal diagnosis, as is well known in the art.

According to some implementations, the evaluation of structural variation may be used to inform additional testing and/or methods of diagnosis. For example, upon the calling of a structural variant, additional PGD or prenatal diagnostic testing may be ordered. In some instances, the additional testing may be specific to one or more diseases associated with a called structural variant. In some instances, more invasive procedures may be performed on the subject, particularly if the subject is an embryo or fetus. For example, tissue biopsies may be performed directly on the embryo or fetus in order to perform sequencing of cellular DNA or other diagnostics on the cellular material. Karyotyping may be performed on the subject. In some implementations, the additional testing may be performed substantially concurrently with the determination of ploidy status (at approximately the same level of development). In some implementations, additional testing may be performed on a postponed schedule, allowing for additional development to occur (e.g., for development from an embryo to a fetus and/or after implantation of an embryo via IVF). In some implementations, additional testing may be performed on a born subject (e.g., an infant or child subject) based on the evaluation of structural variation made when the subject was an embryo and/or fetus.

The evaluation of structural variation may be used to inform treatment decisions for the subject. For example, upon the calling of a structural variant, the subject may be treated for a disease or condition associated with the variant. The treatment may comprise any treatment suitable for the subject's stage of development. For example, genetic editing may be performed on an embryo and/or prenatal treatments may be administered to a fetus (or mother carrying the fetus). In some implementations, treatments may be performed on a postponed schedule, allowing for additional development to occur (e.g., for development from an embryo to a fetus and/or after implantation of an embryo via IVF). In some implementations, treatment may be performed on a born subject (e.g., an infant or child subject) based on ploidy status determinations made when the subject was an embryo and/or fetus. The early detection of a structural variant (e.g., while in utero) may allow for earlier treatment in infants and children, which may provide improved outcomes.

### Disease Diagnosis

Structural variants that are identified according to the methods herein may be associated with a pathogenicity or disease or otherwise be determined to be non-pathogenic. Machine learning techniques may be used to evaluate the pathogenicity of a structural variant. In addition to diagnoses described elsewhere herein that are based on known associations of a structural variant (e.g., CNV) with a disease, the methods and/or systems described herein may be used to identify novel associations between structural variants and diseases. By identifying the same structural variant among a population of subjects having a particular disease or disposition for a disease an association between the structural variant and disease may be established.

Upon identification of a structural variant associated with a disease, sequencing may be conducted in other subjects for diagnostic purposes of determining predisposition for the disease. The sequencing may be targeted to capture the structural variant. The sequencing may be conducted to target neighboring SNPs, such as those determined to be in linkage disequilibrium with the structural variant, as described elsewhere herein, (e.g., via microarrays). The sequencing be conducted to target both a structural variant (e.g. a rare structural variant) and SNP (e.g., a common SNP).

Treatment for the disease may be informed based on any of the diagnostic methods described herein. For example, a subject may be treated (including prophylactic treatment) for a disease for which the subject has been diagnosed as having or at least having an increased disposition for having or developing. Diagnosis and treatment may be performed in combination with other clinical factors and variables as is understood in the art.

Various embodiments and aspects of the disclosure are described with reference to details discussed herein, and the accompanying drawings illustrate various embodiments. The specific details of the description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosures.

Reference in the specification to “one embodiment” or “an embodiment” means that a particular feature, structure, or characteristic described in conjunction with the embodiment can be included in at least one embodiment of the disclosure. The appearances of the phrase “in one embodiment” in various places in the specification do not necessarily all refer to the same embodiment.

It should be understood that the disclosure herein contemplates any possible combination of the various embodiments described herein even if not explicitly exemplified, unless indicated otherwise, explicitly or by context (e.g., where various aspects would be understood to be physically incompatible).

Unless specifically stated otherwise, terms such as creating, training, reconstructing, calculating, identifying, or the like, refer to actions and processes performed or implemented by computing devices that manipulates and transforms data represented as physical (electronic) quantities within the computing device's registers and memories into other data similarly represented as physical quantities within the computing device memories or registers or other such information storage, transmission or display devices. Also, the terms “first,” “second,” “third,” “fourth,” etc., as used herein are meant as labels to distinguish among different elements and may not necessarily have an ordinal meaning according to their numerical designation.

Examples described herein also relate to an apparatus for performing the operations described herein. This apparatus may be specially constructed for the required purposes, or it may comprise a general purpose computing device selectively programmed by a computer program stored in the computing device. Such a computer program may be stored in a computer-readable non-transitory storage medium.

The methods and illustrative examples described herein are not inherently related to any particular computer or other apparatus. Various general purpose systems may be used in accordance with the teachings described herein, or it may prove convenient to construct more specialized apparatus to perform the required method steps. The required structure for a variety of these systems will appear as set forth in the description above.

The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

As used herein, the singular forms “a”, “an” and “the” are intended to include the plural forms as well, unless the context clearly indicates otherwise. It will be further understood that the terms “comprises”, “comprising”, “includes”, and/or “including”, when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

### EXAMPLES

### Example 1. Elimination of Candidate Structural Variant in a Noisy Sequencing Region

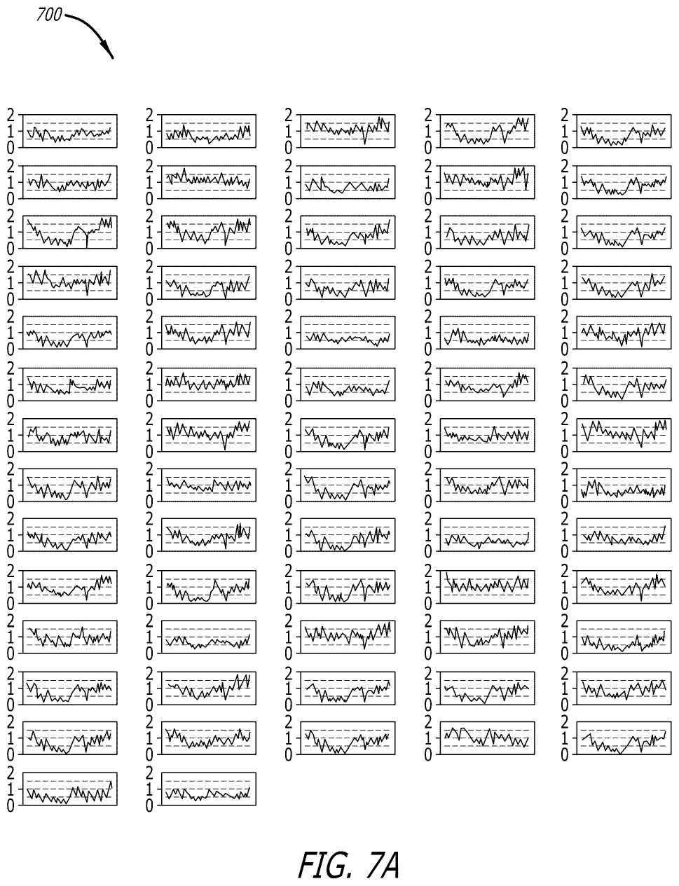

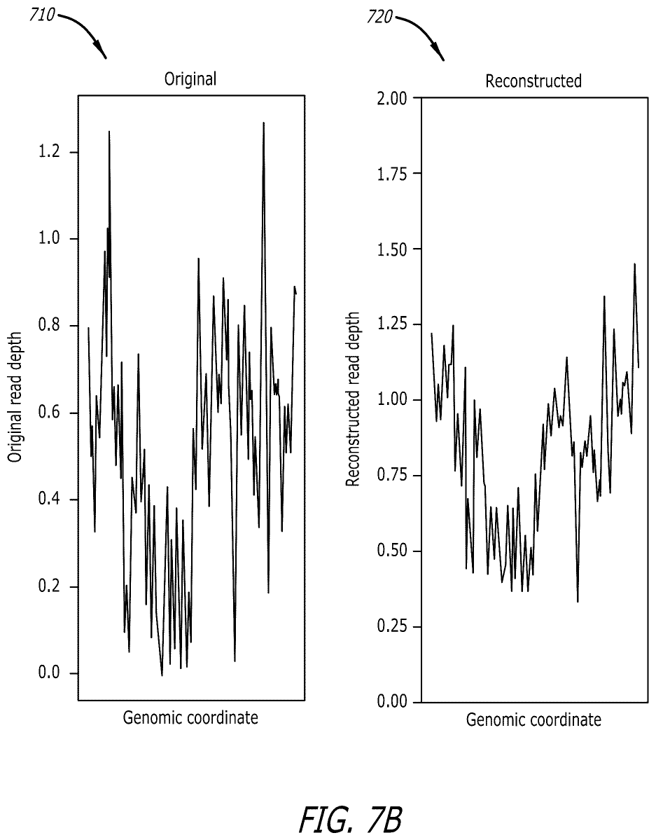

A candidate structural variant was evaluated in the genome of a human subject at chr5: 70475401-70483600. Chr5 indicates the chromosome (human chromosome 5) where the candidate structural variant is located, 70475401 indicates the start nucleotide coordinate of the candidate structural variant within the chromosome, and 70483600 indicates the ending nucleotide coordinate of the candidate structural variant within the chromosome. The candidate structural variant was evaluated with an autoencoder built according to one embodiment to determine if the candidate structural variant actually existed in the subject's genome. The autoencoder was trained on training input 700 comprising 67 read depth profiles spanning the candidate structural variant obtained from reference genetic samples. The read depth profiles of training input 700 are represented by the read depth graphs depicted in FIG. 7A. The trained autoencoder was then provided an input 710 of an original read depth profile for chr5: 70475401-70483600 obtained from sequencing data for the subject and an output 720 comprising a reconstructed read depth profile was generated using the autoencoder. The original and reconstructed read depth profiles 710, 720 are represented by the read depth graphs depicted in FIG. 7B. Reconstruction errors were determined for each data point of the input 710 and output 720 read depth profiles and a mean squared error of 0.9 was calculated. The mean squared error was determined to be below a score threshold selected to be indicative of sufficient dissimilarity between the original and reconstructed read depth profiles to identify the presence of a true structural variant. Accordingly, the candidate structural variant in the subject was classified as a false positive or as normal read depth variation instead of an anomaly and a real structural variant was not called. The read depth profile of the region comprising the candidate structural variant was manually/visually inspected and the sequencing was confirmed to appear noisy. This example demonstrates that the autoencoder was able to account for noise in the sequencing data and accurately reconstruct the read depth profile of the subject, because the subject's read depth profile was similar to those of the reference samples, which did not comprise the candidate structural variant.

### Example 2. Identification of Candidate Structural Variant as Rare Deletion

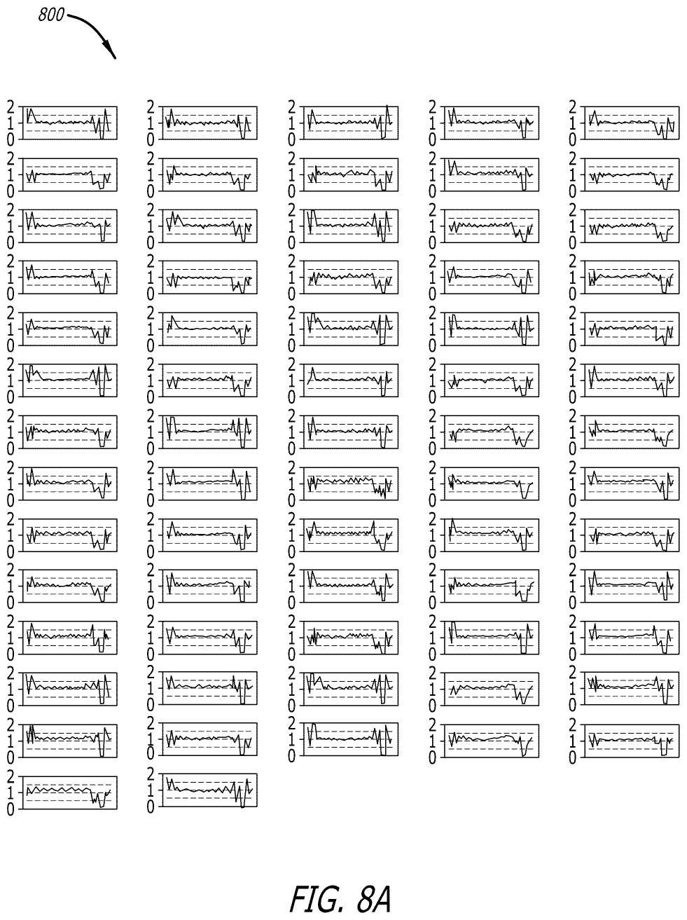

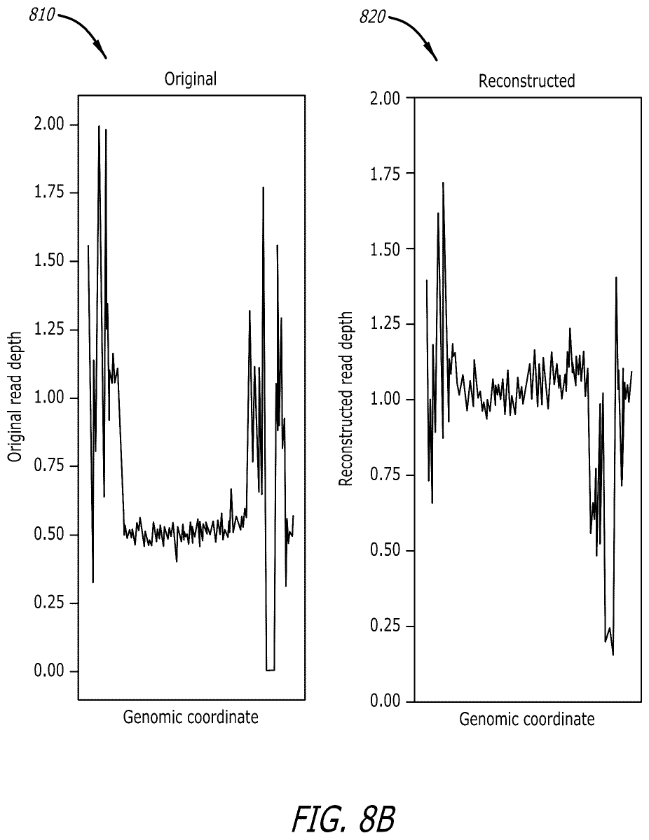

A candidate structural variant was evaluated in the genome of a human subject at chr22: 19015001-20443001. Chr22 indicates the chromosome (human chromosome 22) where the candidate structural variant is located, 19015001 indicates the start nucleotide coordinate of the candidate structural variant within the chromosome, and 20443001 indicates the ending nucleotide coordinate of the candidate structural variant within the chromosome. The candidate structural variant was evaluated with an autoencoder built according to one embodiment to determine if the candidate structural variant actually existed in the subject's genome. The autoencoder was trained on training input 800 comprising 67 read depth profiles spanning the candidate structural variant obtained from reference genetic samples. The read depth profiles of training input 800 are represented by the read depth graphs depicted in FIG. 8A. The trained autoencoder was then provided an input 810 of an original read depth profile for chr22: 19015001-20443001 obtained from sequencing data for the subject and an output 820 comprising a reconstructed read depth profile was generated using the autoencoder. The original and reconstructed read depth profiles 810, 820 are represented by the read depth graphs depicted in FIG. 8B. Reconstruction errors were determined for each data point of the input 810 and output 820 read depth profiles and a mean squared error of 4.23 was calculated. The mean squared error was determined to be above a cutoff score threshold selected to be indicative of sufficient dissimilarity between the original and reconstructed read depth profiles to identify the presence of a true structural variant. Accordingly, the candidate structural variant in the subject was called as a deletion. This example demonstrates that because the autoencoder was not trained on read depth profiles with similar deletions, it was unable to accurately reconstruct the read depth profile of a subject having such a deletion.

### Example 3. Calling a Duplication Using a Distribution of Reconstruction Errors

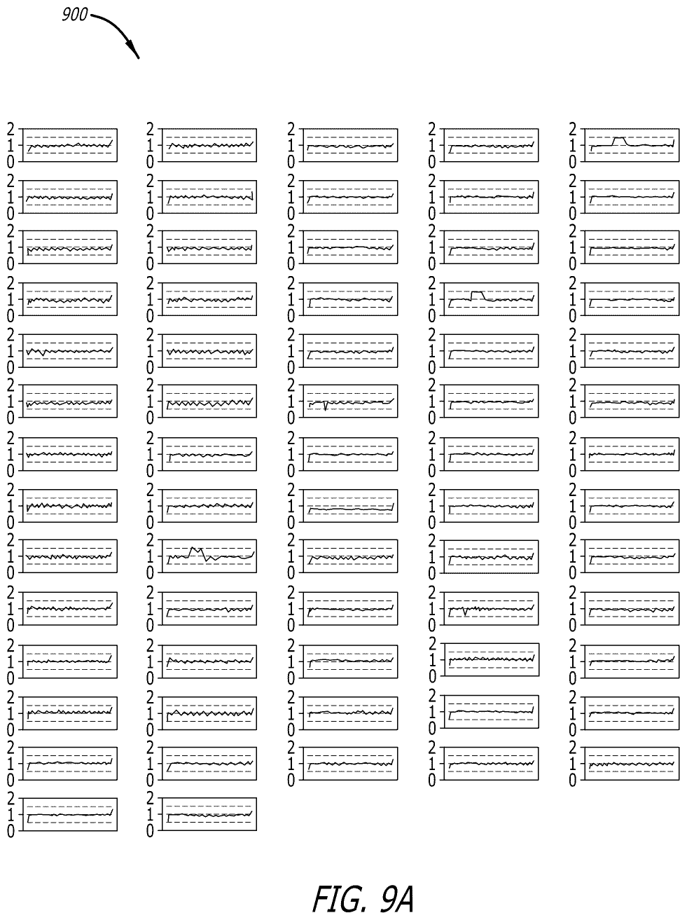

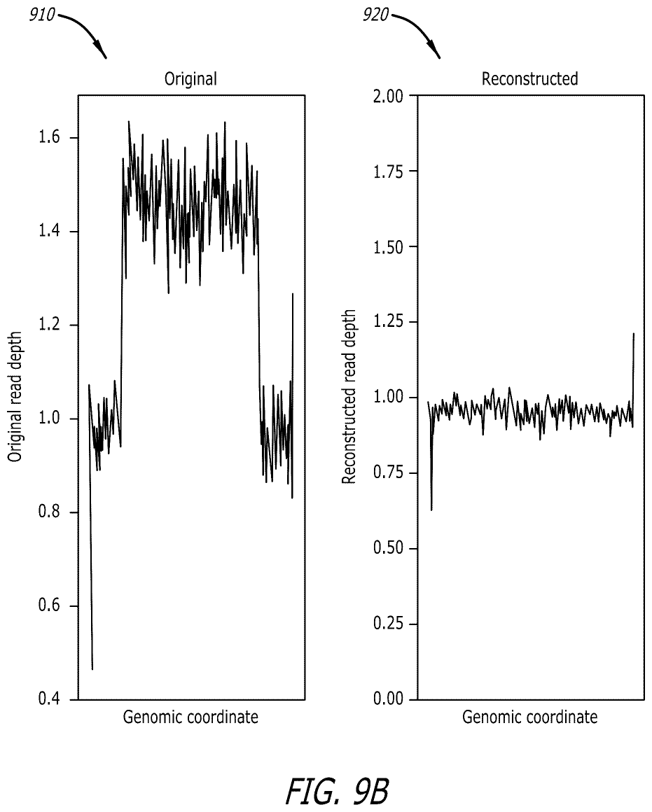

A candidate structural variant was evaluated in the genome of a human subject at chr6: 162557549-163124862. Chr6 indicates the chromosome (human chromosome 6) where the candidate structural variant is located, 162557549 indicates the start nucleotide coordinate of the candidate structural variant within the chromosome, and 163124862 indicates the ending nucleotide coordinate of the candidate structural variant within the chromosome. The candidate structural variant was evaluated with an autoencoder built according to one embodiment to determine if the candidate structural variant actually existed in the subject's genome. The autoencoder was trained on training input 900 comprising 67 read depth profiles spanning the candidate structural variant obtained from reference genetic samples. The read depth profiles of training input 900 are represented by the read depth graphs depicted in FIG. 9A. The trained autoencoder was then provided an input 910 of an original read depth profile for chr6: 162557549-163124862 obtained from sequencing data for the subject and an output 920 comprising a reconstructed read depth profile was generated using the autoencoder. The original and reconstructed read depth profiles 910, 920 are represented by the read depth graphs depicted in FIG. 9B. The trained autoencoder was also provided inputs of original read depth profiles for chr6: 162557549-163124862 obtained from 29 different reference genetic samples and the autoencoder was used to reconstruct read depth profiles for each. Reconstruction errors were determined for each data point of the input 910 and output 920 read depth profiles as well as the 29 different reference genetic samples and a mean squared error was calculated for each, shown in Table 1 below. The mean and standard deviation of the reconstruction error distribution for the set of 30 analyzed read depth was then determined so that a z-score could be calculated for each input profile, as shown in Table 1. The subject was determined to have a z-score of 13.05, which is above a score threshold of 2.0 indicative of sufficient dissimilarity between the original and reconstructed read depth profiles to identify the presence of a true structural variant. Accordingly, the candidate structural variant in the subject was called as a duplication. This example demonstrates how reference samples may be used to calibrate appropriate score thresholds for calling structural variants.

TABLE 1
Distribution of Autoencoder Reconstruction Errors

| | Reconstruction | |
|---|---|---|
| | Error (Mean- | |
| Sample ID | sequence error) | Z-score |
| Subject with candidate | 5.38 | 13.05 |
| duplication | | |
| Reference sample 1 | −0.07 | 0.5 |
| Reference sample 2 | −0.14 | 0.32 |
| Reference sample 3 | −0.14 | 0.33 |
| Reference sample 4 | −0.15 | 0.3 |
| Reference sample 5 | −0.15 | 0.31 |
| Reference sample 6 | −0.16 | 0.28 |
| Reference sample 7 | −0.17 | 0.27 |
| Reference sample 8 | −0.17 | 0.27 |
| Reference sample 9 | −0.17 | 0.27 |
| Reference sample 10 | −0.18 | 0.24 |
| Reference sample 11 | −0.18 | 0.23 |
| Reference sample 12 | −0.18 | 0.24 |
| Reference sample 13 | −0.19 | 0.2 |
| Reference sample 14 | −0.2 | 0.18 |
| Reference sample 15 | −0.2 | 0.19 |
| Reference sample 16 | −0.2 | 0.2 |
| Reference sample 17 | −0.2 | 0.18 |
| Reference sample 18 | −0.2 | 0.19 |
| Reference sample 19 | −0.2 | 0.18 |
| Reference sample 20 | −0.2 | 0.18 |
| Reference sample 21 | −0.2 | 0.2 |
| Reference sample 22 | −0.21 | 0.18 |
| Reference sample 23 | −0.21 | 0.16 |
| Reference sample 24 | −0.21 | 0.17 |
| Reference sample 25 | −0.21 | 0.18 |
| Reference sample 26 | −0.21 | 0.16 |
| Reference sample 27 | −0.22 | 0.13 |
| Reference sample 28 | −0.22 | 0.16 |
| Reference sample 29 | −0.23 | 0.11 |

### Example 4. Evaluation of Autoencoder Performance on a Large Number of Structural Variants

Three test datasets having candidate structural variants that were known to be either true or false variants were used to test the performance of an autoencoder as described in Example 3 over a large number of candidate structural variants. The first dataset (“Pathogenic SVs”) consisted of 22 pathogenic SVs from clinical samples. The second dataset (“Rare SVs”) consisted of 668 SVs identified in subjects that overlap with SVs in the Gnomad database with minor allele frequency less than 0.01. The third dataset “True Negative SVs” consisted of 440 SVs identified in clinical subjects that were manually inspected and deemed not to be real. Any structural variants that were ambiguous after visual inspection were discarded and not included in any of the three datasets.

The three datasets were each also evaluated with two alternative methods for computationally calling structural variants using the same reference samples used to train and test the autoencoder in order to provide a comparison to the autoencoder. The first alternative method (“Alternative Method 1”) calculates an average read depth for each read depth profile from reference samples and then a standard deviation from the average read depths. The SV is classified as rare and real if the subject's average read depth is greater than or equal to +/−2 standard deviations. The second alternative method (“Alternative Method 2”) calculates average read depth per position for all reference samples to create an average read depth profile. The mean squared errors of subject and reference test samples are calculated against the average read depth profile. A z-score for the subject is calculated using the distribution of mean squared errors from the reference samples. The SV is positively identified if the z-score is greater than or equal to 2 standard deviations.

Table 2 below shows the sensitivity (true positive rate) for each method detection of the true variants and the specificity (true negative rate) for each method's detection of false variants.

TABLE 2
Autoencoder accuracy compared to alternative methods

| | Sensitivity | Sensitivity of | Specificity of |
|---|---|---|---|
| | of Clinical | | True |
| Method | Samples | Rare SVs | Negative SVs |
| Autoencoder | 100% (22/22) | 99.85% | 87.5% (385/440) |
| | | (667/668) | |
| Alternative | 100% (22/22) | 99.4% (664/668) | 88.2% (388/440) |
| Method 1 | | | |
| Alternative | 100% (22/22) | 98.7% (659/668) | 86.14% (379/440) |
| Method 2 | | | |

### CLAIM CLAUSES

Clause 1. A computer-implemented method for structural variation identification using an autoencoder, the computer-implemented method comprising: obtaining an original read depth profile for a candidate structural variant region of a sample obtained from a subject having a candidate structural variant; generating a reconstructed read depth profile for the candidate structural variant region of the sample using the autoencoder; calculating a score, wherein the score is calculated based at least in part on differences between the reconstructed read depth profile and the original read depth profile; determining whether the score satisfies a score threshold; and in an instance in which the score satisfies the score threshold, reporting the candidate structural variant as real.
Clause 2. The computer-implemented method of clause 1, further comprising, prior to generating the reconstructed read depth profile, training the autoencoder using one or more reference samples.
Clause 3. The computer-implemented method any of clauses 1 to 2, wherein (i) the one or more reference samples share a particular label and (ii) sharing a common label comprises sharing at least one common characteristic.
Clause 4. The computer-implemented method any of clauses 1 to 3, wherein the at least one common characteristic comprises one or more of a disease status, membership in a specific reference population, or a sample collection type.
Clause 5. The computer-implemented method any of clauses 1 to 4, wherein the one or more reference samples are derived from non-tumor samples.
Clause 6. The computer-implemented method of any of clauses 1 to 5, wherein the original read depth profile is generated based on sequencing data associated with the sample obtained from the subject.
Clause 7. The computer-implemented method of any of clauses 1 to 6, wherein the sample is derived from a tumor.
Clause 8. The computer-implemented method of any of clauses 1 to 7, further comprising: generating a secondary reconstructed read depth profile using a secondary autoencoder, wherein the autoencoder is associated with a first label and the secondary autoencoder is associated with a second label that is different from the first label; calculating a secondary score, wherein the secondary score is calculated based on a difference or deviation between the secondary reconstructed read depth profile and the original read depth profile; selecting either the first label or second label based on a comparison of the score and the secondary score to an ideal score; in an instance in which the first label is selected and the score satisfies the score threshold, reporting the candidate structural variant as real and with the first label; and in an instance in which the second label is selected and the secondary score satisfies the score threshold, reporting the candidate structural variant as real and with the second label.
Clause 9. The computer-implemented method of any of clauses 1 to 8, further comprising: training the secondary autoencoder using one or more reference samples that are labeled differently from the one or more reference samples used to train the first autoencoder.
Clause 10. The computer-implemented method of any of clauses 1 to 9, wherein the read depth profiles includes regions flanking the candidate structural variant.
Clause 11. The computer-implemented method of any of clauses 1 to 10, wherein the read depth profiles span breakpoints of the candidate structural variant.
Clause 12. The computer-implemented method of any of clauses 1 to 11, wherein the read depth profiles comprise a subset of chromosomal positions in or near the candidate structural variant region.
Clause 13. The computer-implemented method any of clauses 1 to 12, wherein the read depth profiles comprise a mean, median, or mode of read depths across a window.
Clause 14. The computer-implemented method of any of clauses 1 to 13, wherein the autoencoder is at least one of (i) a sparse autoencoder, (ii) a convolutional neural network, (iii) a regularized autoencoder, or (iv) a variational autoencoder.
Clause 15. The computer-implemented method of any of clauses 1 to 14, wherein the score is a reconstruction error and calculating the score further comprises: determining the reconstruction error based on a mean squared error of one or more data points of the original read depth profile and one more corresponding data points of the reconstructed read depth profile.
Clause 16. The computer-implemented method of any of clauses 1 to 15, wherein the score is a z-score and calculating the score further comprises: determining a reconstruction error; and calculating a z-score for the reconstruction error based on a mean and standard deviation of reconstruction errors calculated for a plurality of test samples.
Clause 17. The computer-implemented method of any of clauses 1 to 16, further comprising: determining whether the score is ranked in a predetermined top percentile of scores calculated for a plurality of test samples, wherein the score satisfies the score threshold in an instance in which the score for is ranked in a predetermined top percentile of scores.
Clause 18. The computer-implemented method of any of clauses 1 to 17, wherein the plurality of test samples comprises one or more reference samples.
Clause 19. The computer-implemented method of any of clauses 1 to 18, wherein the candidate structural variant is at least one of: (i) a deletion, (ii) a copy number variant, (iii) an insertion, (iv) an inversion, or (v) a translocation.
Clause 20. An apparatus for structural variation identification using an autoencoder, the apparatus comprising: means for obtaining an original read depth profile for a candidate structural variant region of a sample obtained from a subject having a candidate structural variant; means for generating a reconstructed read depth profile for the candidate structural variant region of the sample using the autoencoder; means for calculating a score, wherein the score is calculated based at least in part on differences between the reconstructed read depth profile and the original read depth profile; means for determining whether the score satisfies a score threshold; and in an instance in which the score satisfies the score threshold, means for reporting the candidate structural variant as real.
Clause 21. The apparatus of clause 20, further comprising, prior to generating the reconstructed read depth profile, means for training the autoencoder using one or more reference samples. Clause 22. The apparatus of any of clauses 20 to 21, wherein (i) the one or more reference samples share a particular label and (ii) sharing a common label comprises sharing at least one common characteristic.
Clause 23. The apparatus of any of clauses 20 to 22, wherein the at least one common characteristic comprises one or more of a disease status, membership in a specific reference population, or a sample collection type.
Clause 24. The apparatus of any of clauses 20 to 23, wherein the one or more reference samples are derived from non-tumor samples.
Clause 25. The apparatus of any of clauses 20 to 24, wherein the original read depth profile is generated based on sequencing data associated with the sample obtained from the subject.
Clause 26. The apparatus of any of clauses 20 to 25, wherein the sample is derived from a tumor.
Clause 27. The apparatus of any of clauses 20 to 26, further comprising: means for generating a secondary reconstructed read depth profile using a secondary autoencoder, wherein the autoencoder is associated with a first label and the secondary autoencoder is associated with a second label that is different from the first label; means for calculating a secondary score, wherein the secondary score is calculated based on a difference or deviation between the secondary reconstructed read depth profile and the original read depth profile; means for selecting either the first label or second label based on a comparison of the score and the secondary score to an ideal score; in an instance in which the first label is selected and the score satisfies the score threshold, means for reporting the candidate structural variant as real and with the first label; and in an instance in which the second label is selected and the secondary score satisfies the score threshold, means for reporting the candidate structural variant as real and with the second label.
Clause 28. The apparatus of any of clauses 20 to 27, further comprising: means for training the secondary autoencoder using one or more reference samples that are labeled differently from the one or more reference samples used to train the first autoencoder.
Clause 29. The apparatus of any of clauses 20 to 28, wherein the read depth profiles includes regions flanking the candidate structural variant.
Clause 30. The apparatus of any of clauses 20 to 29, wherein the read depth profiles span breakpoints of the candidate structural variant.
Clause 31. The apparatus of any of clauses 20 to 30, wherein the read depth profiles comprise a subset of chromosomal positions in or near the candidate structural variant region. Clause 32. The apparatus of any of clauses 20 to 31, wherein the read depth profiles comprise a mean, median, or mode of read depths across a window.
Clause 33. The apparatus of any of clauses 20 to 32, wherein the autoencoder is at least one of (i) a sparse autoencoder, (ii) a convolutional neural network, (iii) a regularized autoencoder, or (iv) a variational autoencoder.
Clause 34. The apparatus of any of clauses 20 to 33, wherein the score is a reconstruction error and calculating the score further comprises: means for determining the reconstruction error based on a mean squared error of one or more data points of the original read depth profile and one more corresponding data points of the reconstructed read depth profile.
Clause 35. The apparatus of any of clauses 20 to 34, wherein the score is a z-score and calculating the score further comprises: means for determining a reconstruction error; and means for calculating a z-score for the reconstruction error based on a mean and standard deviation of reconstruction errors calculated for a plurality of test samples.
Clause 36. The apparatus of any of clauses 20 to 35, further comprising: means for determining whether the score is ranked in a predetermined top percentile of scores calculated for a plurality of test samples, wherein the score satisfies the score threshold in an instance in which the score for is ranked in a predetermined top percentile of scores.
Clause 37. The apparatus of any of clauses 20 to 36, wherein the plurality of test samples comprises one or more reference samples.
Clause 38. The apparatus of any of clauses 20 to 37, wherein the candidate structural variant is at least one of: (i) a deletion, (ii) a copy number variant, (iii) an insertion, (iv) an inversion, or (v) a translocation.
Clause 39. A computer program product for structural variation identification using an autoencoder, the computer program product comprising at least one non-transitory computer-readable storage medium storing software instructions that, when executed, cause an apparatus to: obtain an original read depth profile for a candidate structural variant region of a sample obtained from a subject having a candidate structural variant; generate a reconstructed read depth profile for the candidate structural variant region of the sample using the autoencoder; calculate a score, wherein the score is calculated based at least in part on differences between the reconstructed read depth profile and the original read depth profile; determine whether the score satisfies a score threshold; and in an instance in which the score satisfies the score threshold, report the candidate structural variant as real.
Clause 40. The computer program product of clause 39, wherein the software instructions, when executed, further cause the apparatus to, prior to generating the reconstructed read depth profile, train the autoencoder using one or more reference samples.
Clause 41. The computer program product of any of clauses 39 to 40, wherein (i) the one or more reference samples share a particular label and (ii) sharing a common label comprises sharing at least one common characteristic.
Clause 42. The computer program product of any of clauses 39 to 41, wherein the at least one common characteristic comprises one or more of a disease status, membership in a specific reference population, or a sample collection type.
Clause 43. The computer program product of any of clauses 39 to 42, wherein the one or more reference samples are derived from non-tumor samples.
Clause 44. The computer program product of any of clauses 39 to 43, wherein the original read depth profile is generated based on sequencing data associated with the sample obtained from the subject.
Clause 45. The computer program product of any of clauses 39 to 44, wherein the sample is derived from a tumor.
Clause 46. The computer program product of any of clauses 39 to 45, wherein the software instructions, when executed, further cause the apparatus to: generate a secondary reconstructed read depth profile using a secondary autoencoder, wherein the autoencoder is associated with a first label and the secondary autoencoder is associated with a second label that is different from the first label; calculate a secondary score, wherein the secondary score is calculated based on a difference or deviation between the secondary reconstructed read depth profile and the original read depth profile; select either the first label or second label based on a comparison of the score and the secondary score to an ideal score; in an instance in which the first label is selected and the score satisfies the score threshold, report the candidate structural variant as real and with the first label; and in an instance in which the second label is selected and the secondary score satisfies the score threshold, report the candidate structural variant as real and with the second label.
Clause 47. The computer program product of any of clauses 39 to 46, wherein the software instructions, when executed, further cause the apparatus to: train the secondary autoencoder using one or more reference samples that are labeled differently from the one or more reference samples used to train the first autoencoder.
Clause 48. The computer program product of any of clauses 39 to 47, wherein the read depth profiles includes regions flanking the candidate structural variant.
Clause 49. The computer program product of any of clauses 39 to 48, wherein the read depth profiles span breakpoints of the candidate structural variant.
Clause 50. The computer program product of any of clauses 39 to 49, wherein the read depth profiles comprise a subset of chromosomal positions in or near the candidate structural variant region.
Clause 51. The computer program product of any of clauses 39 to 50, wherein the read depth profiles comprise a mean, median, or mode of read depths across a window.
Clause 52. The computer program product of any of clauses 39 to 51, wherein the autoencoder is at least one of (i) a sparse autoencoder, (ii) a convolutional neural network, (iii) a regularized autoencoder, or (iv) a variational autoencoder.
Clause 53. The computer program product of any of clauses 39 to 52, wherein the score is a reconstruction error and wherein the software instructions, when executed, further cause the apparatus to: determine the reconstruction error based on a mean squared error of one or more data points of the original read depth profile and one more corresponding data points of the reconstructed read depth profile.
Clause 54. The computer program product of any of clauses 39 to 53, wherein the score is a z-score and wherein the software instructions, when executed, further cause the apparatus to: determine a reconstruction error; and calculate a z-score for the reconstruction error based on a mean and standard deviation of reconstruction errors calculated for a plurality of test samples.
Clause 55. The computer program product of any of clauses 39 to 54, wherein the software instructions, when executed, further cause the apparatus to: determine whether the score is ranked in a predetermined top percentile of scores calculated for a plurality of test samples, wherein the score satisfies the score threshold in an instance in which the score for is ranked in a predetermined top percentile of scores.
Clause 56. The computer program product of any of clauses 39 to 55, wherein the plurality of test samples comprises one or more reference samples.
Clause 57. The computer program product of any of clauses 39 to 56, wherein the candidate structural variant is at least one of: (i) a deletion, (ii) a copy number variant, (iii) an insertion, (iv) an inversion, or (v) a translocation.

What is claimed is:

1. A computer-implemented method for structural variation identification using a machine-learning model, the computer-implemented method comprising:
obtaining an original read depth profile for a candidate structural variant region of a sample obtained from a subject having a candidate structural variant;
generating a reconstructed read depth profile for the candidate structural variant region of the sample using a machine-learning model;
calculating a score, wherein the score is calculated based at least in part on differences between the reconstructed read depth profile and the original read depth profile;
determining whether the score satisfies a score threshold; and
in an instance in which the score satisfies the score threshold, reporting the candidate structural variant as real.

2. The computer-implemented method of claim 1, wherein the machine-learning model comprises a neural network, deep neural network (DNN), a convolutional neural network, or an autoencoder.

3. The computer-implemented method of claim 2, wherein the machine-learning model comprises a neural network, and wherein the neural network comprises one or more encoder layers, one or more hidden layers, and one or more decoder layers.

4. The computer-implemented method of claim 1, wherein the score is a z-score and calculating the score further comprises:
determining a reconstruction error; and
calculating a z-score for the reconstruction error based on a mean and standard deviation of reconstruction errors calculated for a plurality of test samples.

5. The computer-implemented method of claim 1, further comprising:
determining whether the score is ranked in a predetermined top percentile of scores calculated for a plurality of test samples,
wherein the score satisfies the score threshold in an instance in which the score for is ranked in a predetermined top percentile of scores.

6. The computer-implemented method of claim 5, wherein the plurality of test samples comprises one or more reference samples.

7. The computer-implemented method of claim 1, wherein the candidate structural variant is at least one of: (i) a deletion, (ii) a copy number variant, (iii) an insertion, (iv) an inversion, or (v) a translocation.

8. The computer-implemented method of claim 1, further comprising, prior to generating the reconstructed read depth profile, training the autoencoder using one or more reference samples and unsupervised learning.

9. The computer-implemented method of claim 8, wherein (i) the one or more reference samples share a particular label and (ii) sharing a common label comprises sharing at least one common characteristic.

10. The computer-implemented method of claim 9, wherein the at least one common characteristic comprises one or more of a disease status, membership in a specific reference population, or a sample collection type.

11. The computer-implemented method of claim 8, wherein the one or more reference samples are derived from non-tumor samples.

12. The computer-implemented method of claim 1, wherein the original read depth profile is generated based on sequencing data associated with the sample obtained from the subject, and wherein the sample is derived from a tumor.

13. The computer-implemented method of claim 1, further comprising:
generating a secondary reconstructed read depth profile using a secondary autoencoder, wherein the autoencoder is associated with a first label and the secondary autoencoder is associated with a second label that is different from the first label;
calculating a secondary score, wherein the secondary score is calculated based on a difference or deviation between the secondary reconstructed read depth profile and the original read depth profile;
selecting either the first label or second label based on a comparison of the score and the secondary score to an ideal score;
in an instance in which the first label is selected and the score satisfies the score threshold, reporting the candidate structural variant as real and with the first label; and
in an instance in which the second label is selected and the secondary score satisfies the score threshold, reporting the candidate structural variant as real and with the second label.

14. The computer-implemented method of claim 1, wherein the read depth profiles includes regions flanking the candidate structural variant.

15. The computer-implemented method of claim 1, wherein the read depth profiles span breakpoints of the candidate structural variant.

16. The computer-implemented method of claim 1, wherein the read depth profiles comprise a subset of chromosomal positions in or near the candidate structural variant region.

17. The computer-implemented method of claim 1, wherein the read depth profiles comprise a mean, median, or mode of read depths across a window.

18. The computer-implemented method of claim 1, wherein the score is a reconstruction error and calculating the score further comprises:
determining the reconstruction error based on a mean squared error of one or more data points of the original read depth profile and one more corresponding data points of the reconstructed read depth profile.

19. A system for structural variation identification using a machine-learning model, the system comprising:
one or more processing devices;
one or more computer-readable storage media storing instructions that, when executed by the one or more processing devices, cause the system to perform operations including:
obtaining an original read depth profile for a candidate structural variant region of a sample obtained from a subject having a candidate structural variant;
generating a reconstructed read depth profile for the candidate structural variant region of the sample using a machine-learning model;
calculating a score, wherein the score is calculated based at least in part on differences between the reconstructed read depth profile and the original read depth profile;
determining whether the score satisfies a score threshold; and
in an instance in which the score satisfies the score threshold, reporting the candidate structural variant as real.

20. A computer program product for structural variation identification using a machine-learning model, the computer program product comprising at least one non-transitory computer-readable storage medium storing software instructions that, when executed, cause an apparatus to perform operations including:
obtaining an original read depth profile for a candidate structural variant region of a sample obtained from a subject having a candidate structural variant;
generating a reconstructed read depth profile for the candidate structural variant region of the sample using the machine-learning model;
calculating a score, wherein the score is calculated based at least in part on differences between the reconstructed read depth profile and the original read depth profile;
determining whether the score satisfies a score threshold; and
in an instance in which the score satisfies the score threshold, reporting the candidate structural variant as real.
